(19) United States
(12) Patent Application Publication
(10) Pub. No.: US 2008/0113044 A1
(43) Pub. Date: May 15, 2008

(54) Extracts and Methods Comprising Green Tea Species

(76) Inventors: Randall S. Alberte, Estero, FL (US); Robert T. Gow, Naples, FL (US); George W. Sypert, Naples, FL (US); Dan Li, Singapore (SG)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/690,613

(22) Filed: Mar. 23, 2007

Related U.S. Application Data
(60) Provisional application No. 60/785,178, filed on Mar. 23, 2006.

(51) Int. Cl. A61K 36/82; A23F 3/16; A23F 3/20; A23F 3/30

(52) U.S. Cl. 424/729; 426/416; 426/428; 426/429; 426/655

(57) ABSTRACT

The present invention relates to extracts of green tea species plant material prepared by supercritical CO₂ extractions methods.

### RELATED APPLICATIONS

This application claims the benefit of priority to U.S. Provisional Patent Application Ser. No. 60/785,178, filed Mar. 23, 2006, which is hereby incorporated by reference in its entirety.

### FIELD OF INVENTION

The invention relates to extracts of green tea species, methods of preparing them using sequential extractions steps, and methods of treatment thereof.

### BACKGROUND OF THE INVENTION

Tea originated in southern China some 4000 years ago and is consumed by over two-thirds of the world's population. Tea has an attractive odor, excellent taste, and health promoting effects making it the most popular beverage in the world, second only to water. As early as 3000 B.C., tea was used by the Chinese as a medicinal drink. The medical use of tea was recorded in the ancient Chinese pharmacopoeia “Ben Cao Gang Mo” written during the Ming dynasty (16^{th} century). The source of tea is the botanical, Camellia sinensis. Literally hundreds of teas are now produced from the leaves of C. sinensis and are generally classified into three major categories: non-fermented green tea, partially fermented oolong, and fully fermented black tea.

Camellia sinesis, a member of the Theaceae family, is an evergreen shrub or tree that can grow to a height of 30 feet. However, it is usually clipped to a height of 1-5 feet in cultivation for tea leaves. The plant is heavily branched with dark-green, hairy, oblong-ovate leaves cultivated and preferentially picked as young shoots. Older leaves are generally considered to be of inferior quality.

Although both green and black teas are derived from the botanical, Camellia, sinensis, it is the processing of the leaves that differentiates the two types of tea. In the case of the black teas, after the leaves are picked, they are permitted to wilt and then rolled. These leaves are allowed to ferment, converting the tea polyphenols (catechins) to phlobaphenes and forming aromatic rings. Fermentation occurs as leaf enzymes, including polyphenol oxidate, reacts with the tea polyphenols, particularly the catechins [1]. In the case of green tea production, the young leaves are not permitted to oxidize. Instead, the leaves are steamed, which inactivates the oxidative enzymes, thus preserving the tea catechins.

The chemical constituents of green tea leaf include the polyphenols, methylxanthines, amino acids, organic acids, carbohydrates, proteins, lignin, lipids, chlorophyll and other pigments, ash, and essential oils, see Table 1 [2,3]. From a commercial and biological standpoint, the polyphenols and caffeine have been traditionally considered to be of greater importance than the other constituents. However, other chemical constituents such as theanine, the essential oils and, the water soluble-ethanol insoluble polysaccharides have recently been shown to have important biologically beneficially effects (see summary below).

TABLE 1
Principle chemical constituents of green tea leaves.

| Chemical constituents | % dry weight |
|---|---|
| Essential Oil Fraction (primarily volatile oils) | 0.1 |
| Polyphenols | 39.0 |
| Catechins | 25.0 |
| Catechin (C) | (0.2) |
| Epicatechin (EC) | (2.2) |
| Epicatechin gallate (ECG) | (1.9) |
| Gallocatechin (GC) | (8.7) |
| Epigallocatechin gallate (EGCG) | (10.9) |
| Epigallocatechin (EGC) | (9.7) |
| Caffeic Acid Derivatives | trace |
| Caffeic acid | |
| Chlorogenic acid | |
| Flavonols & flavonol glycosides | 3.0 |
| Quercetin | (0.4) |
| Rutin | (1.5) |
| Kaempferol | (0.5) |
| Other phenolic acids (tannins) | 12.0 |
| Methylxanthines | 3.5 |
| Caffeine* | 3.3 |
| Theobromine | 0.1 |
| Amino Acids | 4.0 |
| Theanine | 0.6 |
| Oxalic Acid* | 0.6 |
| Polysaccharides | 13.0 |
| Monosaccharides | 4.0 |
| Cellulose | 7.0 |
| Protein | 15.0 |
| Organic acids | 0.5 |
| Lignin | 6.0 |
| Lipids | 3.0 |
| Chlorophyll & other pigments | 0.5 |
| Ash | 5.0 |

*Toxicity

Green tea contains 30-42% polyphenols by % mass dry weight. The majority of these polyphenols which also have been reported to have the greatest biologically beneficial activity are the flavonols know as “catechins”. The principal catechins Include the following: (−)-epigallocatechin-3-gallate (EGCG), (−)-epigallocatechin (EGC), (−)-catechin gallate (CG), and epicatechin (EC), The highest concentrations is in the order of EGCG followed by EGC, ECG, EC in decreasing order. Other catechins including (+)-gallocatechin (GC), (−)-gallocatechin gallate (GCG), (−)-catechin gallate (CG), and (+)-catechin (C) are present in minor quantities. Many beneficial biological effects of the catechins have been studied. They include anti-oxidative activities, antimutagenic effects, anti-carcinogenic effects, nitrosation inhibition, and inhibitory actions of growth of tumor and immortalized cells but no effect on normal cells. However other chemical constituent groups also exhibit biologically beneficial effects. For example, the essential oil (EO) chemical constituents have anti-oxidant activity, anti-asthmatic activity, anti-bacterial activity, anti-viral activity, anti-cancer activity, immunological enhancement activity, hypoglycemic activity, hypolipidemic activity, anti-inflammatory activity, anti-dermatitic activity, anti-acne activity, and anti-atherosclerosis activity. Theanine (T) has anxiety reducing and mood enhancing activity, cognitive enhancing activity, anti-cancer activity, neuroprotective against cerebral ischemia and stroke, and weight reduction activity. Furthermore, the green tea polysaccharides (P) have anti-oxidant and oxygen free radical scavenging activity, anti-diabetic activity and immunological enhancing activity.

To briefly summarize the therapeutic value of green tea's chemical constituents, recent scientific research and clinical studies have demonstrated the following therapeutic effects of the various chemical compounds, chemical fractions, and gross extraction products of green tea which include the following: powerful anti-oxidant, oxygen free radical scavenging, and nitrosation inhibition (EO, catechins-primarily ECGC & ECG, P, extract) [4-7]; anti-mutagenic activity (EO, catechins, extract) [7-12]; anti-carcinogenic activity without effect on normal cells (EO, catechins, T, extract) [7-13]; skin protective (EO, catechins, P, extract) [8, 10, 11, 14, 15]; anti-cardiovascular disease (EO, catechins, extract) [4-7,16,17]; anti-hyperlipidemia (extract) [16]; anti-stroke and cerebral protection (EO, catechins, T, P, extract) [18, 19]; anti-periodontal disease (extract) [20]; anti-osteoporosis (extract) [21]; immune enhancement (extract) [22]; anti-viral, anti-HIV, and anti-bacterial (EO, catechins, extract) [23]; weight loss and thermogenesis (catechins, caffeine, T, extract) [23,24]; anti-aging (catechins-ECGC, extract) [23]; anxiety reduction, mood enhancer, and cognitive enhancer (T, extract) [25,26]; and anti-diabetes (P, extract) [27].

Although green tea is generally safe and not toxic at very high doses, one potential outcome of consumption of green tea beverages and medicinal products is the development of caffeine related disorders such as cardiac arrhythmias, gastrointestinal disorders, and caffeine toxicity manifested by jitteriness, generalized anxiety, insomnia. Moreover, excessive consumption of caffeine exaggerates stress and stress-related hormone release. Blood pressure may be elevated and the risks of heart attack and stroke are increased when excessive caffeine is consumed.

In view of the lack of comprehensive selectivity in currently available extraction processes, presently available green tea products are suspect regarding their chemical compositions. Thus, what is needed are novel and reproducible green tea extract compositions that combine purified essential oil, catechins with high ECGC, theanine, and polysaccharide chemical constituent fractions with low caffeine concentrations that can be produced with standardized and reliable amounts of these synergistically [14,28] acting physiologically and medically beneficial green tea chemical constituents.

### SUMMARY OF THE INVENTION

In one aspect, the present invention relates to a green tea species extract comprising a fraction having a Direct Analysis in Real Time (DART) mass spectrometry chromatogram of any of FIGS. 6 to 25.

In a further embodiment, the extract comprises a compound selected from the group consisting of an essential oil, a polyphenol, a polysaccharide, and combinations thereof. In a further embodiment, the essential oil is selected from the group consisting of n-hexadecanoic acid, tetradecanoic acid, 9-hexadecanol, 1-undecanol, 1-hexadecanol, oleyl alcohol, 9-octadecen-1-ol, nonadecanol, and combinations thereof. In a further embodiment, the polyphenol is selected from the group consisting of catechins, flavanols, flavonol glycosides, and combinations thereof. In a further embodiment, the catechin is selected from the group consisting of catechin (C), epicatechin (EC), epicatechin gallate (ECG), gallocatechin (GC), epigallocatechin gallate (EGCG), epigallocatechin (EGC), and combinations thereof. In a further embodiment, the flavanol is selected from the group consisting of quercetin and rutin. In a further embodiment, the flavonol glycoside is kaempferol. In a further embodiment, the polysaccharide is selected from the group consisting of glucose, arabinose, galactose, rhamnose, xylose uronic acid and combinations thereof. In a further embodiment, the green tea species of the present invention are substantially free of caffeine, oxalic acid, or tannins.

In a further embodiment, the amount of essential oil is greater than 2% by weight. In a further embodiment, the amount of essential oil is from 25% to 90% by weight. In a further embodiment, the amount of essential oil is from 50% to 90% by weight. In a further embodiment, the amount of essential oil is from 75% to 90% by weight.

In a further embodiment, the amount of polyphenol is greater than 40% by weight. In a further embodiment, the amount of polyphenol is from 50% to 90% by weight. In a further embodiment, the amount of polyphenol is from 75% to 90% by weight.

In a further embodiment, the amount of polysaccharide is greater than 15% by weight. In a further embodiment, the amount of polysaccharide is from 25% to 90% by weight. In a further embodiment, the amount of polysaccharide is from 50% to 90% by weight. In a further embodiment, the amount of polysaccharide is from 75% to 90% by weight.

In a further embodiment, the green tea species extract comprises an essential oil from 2% to 97% by weight, a catechin from 15% to 98% by weight, a theanine from 4% to 90% by weight, and a polysaccharide from 9% to 98% by weight.

In another aspect, the present invention relates to food or medicament comprising the green tea species extract of the present invention.

In another aspect, the present invention relates to a method of preparing a green tea extract having at least one predetermined characteristic comprising sequentially extracting a green tea species plant material to yield an essential oil fraction, a polyphenol fraction, and a polysaccharide fraction by a) extracting a green tea species plant material by super critical carbon dioxide extraction to yield an essential oil fraction and a first residue; b) extracting either a green tea species plant material or the first residue from step a) by alcoholic extraction to yield the polyphenolic fraction and a second residue; and c) extracting the second residue from step a) by water extraction and precipitating the polysaccharide with alcohol to yield the polysaccharide fraction.

In a further embodiment, the first residue from step a) is further decaffeinated by supercritical carbon dioxide extraction. In a further embodiment, the polyphenolic fraction is further purified by affinity adsorbent chromatography.

In a further embodiment, step a) comprises: 1) loading in an extraction vessel ground green tea species plant material; 2) adding carbon dioxide under supercritical conditions; 3) contacting the green tea species plant material and the carbon dioxide for a time; and 4) collecting an essential oil fraction in a collection vessel. In a further embodiment, step a) further comprises altering the essential oil chemical compound ratios by fractionating the essential oil fraction with a supercritical carbon dioxide fractional separation system. In a further embodiment, supercritical conditions comprise 60 bars to 800 bars of pressure at 35° C. to 90° C. In a further embodiment, supercritical conditions comprise 60 bars to 500 bars of pressure at 40° C. to 80° C. In a further embodiment, the time is 30 minutes to 2.5 hours. In a further embodiment, the time is 1 hour.

In a further embodiment, step b) comprises: 1) contacting ground green tea species plant material or the first residue from step a) with an alcoholic solvent for a time sufficient to extract polyphenol chemical constituents; 2) passing an aqueous solution of extracted polyphenolic chemical constituents from step 1) through an affinity adsorbent resin column wherein the polyphenolic constituents are adsorbed; 3) eluting the caffeine compounds from the affinity adsorbent using an acidic elution solvent; and 4) eluting the polyphenolic chemical constituents from the affinity adsorbent resin using a hydro-alcoholic eluting solvent. In a further embodiment, the hydro-alcoholic solution comprises ethanol and water wherein the ethanol concentration is 10-95% by weight. In a further embodiment, the hydro-alcoholic solution comprises ethanol and water wherein the ethanol concentration is 25% by weight. In a further embodiment, step 1) is carried out at 30° C. to 100° C. In a further embodiment, step 1) is carried out at 60° C. to 100° C. In a further embodiment, the time is 1-10 hours. In a further embodiment, the time is 1-5 hours. In a further embodiment, the time is 2 hours.

In a further embodiment, step c) comprises: 1) contacting the second residue from step b) with water for a time sufficient to extract polysaccharides; and 2) precipitating the polysaccharides from the water solution by alcohol precipitation. In a further embodiment, the water is at 70° C. to 90° C. In a further embodiment, the water is at 80° C. to 90° C. In a further embodiment, the time is 1-5 hours. In a further embodiment, the time is 2-4 hours. In a further embodiment, the time is 2 hours. In a further embodiment, the alcohol is ethanol.

In another aspect, the present invention relates to a green tea species extract prepared by the methods of the present invention.

In another aspect the present invention relates to a green tea species extract comprising pyrogallol, theophylline/theobromine at 25 to 35% by weight of the pyrogallol, shikimic acid at 0.1 to 5% by weight of the pyrogallol, coumaric acid at 0.1 to 5% by weight of the pyrogallol, and 3-methoxy-1-tyrosine at 0.1 to 5% by weight of the pyrogallol.

In another aspect the present invention relates to a green tea species extract comprising theanine, theophylline/theobromine at 20 to 30% by weight of the theanine, catechin/epicatechin at 1 to 10% by weight of the theanine, gallic acid at 1 to 10% by weight of the theanine, catechin quinone at 0.1 to 5% by weight of the theanine, cinnamaldehyde at 0.1 to 5% by weight of the theanine, and 3-methoxy-1-tyrosine at 1 to 10% by weight of the theanine.

In another aspect the present invention relates to a green tea species extract comprising theanine, theophylline/theobromine at 45 to 55% by weight of the theanine, catechin/epicatechin at 1 to 10% by weight of the theanine, carnosic acid at 0.1 to 5% by weight of the theanine, gallic acid at 1 to 10% by weight of the theanine, catechin quinone at 0.5 to 5% by weight of the theanine, cinnamaldehyde at 1 to 10% by weight of the theanine, methyl cinnamic acid at 0.1 to 5% by weight of the theanine, cinnamide at 1 to 10% by weight of the theanine, and 3-methoxy-1-tyrosine at 1 to 10% by weight of the theanine.

In another aspect the present invention relates to a green tea species extract comprising pyrogallol, theophylline/theobromine at 1 to 10% by weight of the pyrogallol, theanine at 0.1 to 5% by weight of the pyrogallol, catechin/epicatechin at 1 to 10% by weight of the pyrogallol, kaempferol at 5 to 15% by weight of the pyrogallol, myricitin at 0.1 to 5% by weight of the pyrogallol, gallocatechin quinone at 0.1 to 5% by weight of the pyrogallol, gallic acid at 65 to 75% by weight of the pyrogallol, catechin quinone at 0.5 to 5% by weight of the pyrogallol, vanillic acid at 1 to 10% by weight of the pyrogallol, and 3-methoxy-1-tyrosine at 1 to 5% by weight of the pyrogallol.

In another aspect the present invention relates to a green tea species extract comprising kaempferol, theanine at 1 to 10% by weight of the kaempferol, catechin/epicatechin at 95 to 105% by weight of the kaempferol, quercetin at 20 to 30% by weight of the kaempferol, myricitin at 5 to 15% by weight of the kaempferol, gallocatechin quinone at 5 to 10% by weight of the kaempferol, gallic acid at 55 to 65% by weight of the kaempferol, catechin quinone at 1 to 10% by weight of the kaempferol, coumaric acid at 10 to 20% by weight of the kaempferol, vanillic acid at 1 to 10% by weight of the kaempferol, and 3-methoxy-1-tyrosine at 15 to 25% by weight of the kaempferol.

In another aspect the present invention relates to a green tea species extract comprising pyrogallol, theophylline/theobromine at 0.5 to 5% by weight of the pyrogallol, catechin/epicatechin at 95 to 105% by weight of the pyrogallol, kaempferol at 55 to 65% by weight of the pyrogallol, quercetin at 20 to 30% by weight of the pyrogallol, myricitin at 10 to 20% by weight of the pyrogallol, gallocatechin quinone at 20 to 30% by weight of the pyrogallol, gallic acid at 50 to 60% by weight of the pyrogallol, catechin quinone at 15 to 25% by weight of the pyrogallol, coumaric acid at 15 to 25% by weight of the pyrogallol, vanillic acid at 1 to 10% by weight of the pyrogallol, and 3-methoxy-1-tyrosine at 0.5 to 5% by weight of the pyrogallol.

In another aspect the present invention relates to a green tea species extract comprising pyrogallol, theophylline/theobromine at 0.5 to 5% by weight of the pyrogallol, catechin/epicatechin at 95 to 105% by weight of the pyrogallol, kaempferol at 55 to 65% by weight of the pyrogallol, quercetin at 20 to 30% by weight of the pyrogallol, myricitin at 10 to 20% by weight of the pyrogallol, gallocatechin quinone at 20 to 30% by weight of the pyrogallol, gallic acid at 50 to 60% by weight of the pyrogallol, catechin quinone at 15 to 25% by weight of the pyrogallol, coumaric acid at 15 to 25% by weight of the pyrogallol, vanillic acid at 1 to 10% by weight of the pyrogallol, and 3-methoxy-1-tyrosine at 0.5 to 5% by weight of the pyrogallol.

In another aspect the present invention relates to a green tea species extract comprising pyrogallol, theanine by weight of the pyrogallol, catechin/epicatechin at 90 to 100% by weight of the pyrogallol, kaempferol at 65 to 75% by weight of the pyrogallol, quercetin at 15 to 25% by weight of the pyrogallol, myricitin at 5 to 15% by weight of the pyrogallol, gallocatechin quinone at 5 to 15% by weight of the pyrogallol, gallic acid at 65 to 75% by weight of the pyrogallol, catechin quinone at 5 to 15% by weight of the pyrogallol, coumaric acid at 10 to 20% by weight of the pyrogallol, vanillic acid at 1 to 10% by weight of the pyrogallol, and 3-methoxy-1-tyrosine at 1 to 10% by weight of the pyrogallol.

The extractions of the present invention are useful in providing physiological and medical effects including, but not limited to, anti-oxidant activity, oxygen free radical scavenging, nitrosation inhibition, anti-mutagenic activity (cancer prevention), anti-carcinogenic activity (cancer therapy), skin protection, anti-aging, anti-cardiovascular disease, anti-stroke disease and therapy, cerebral protection, anti-hyperlipidemia, anti-periodontal disease, anti-osteoporosis, immunological enhancement, anti-viral, anti-HIV and anti-bacterial activity, anti-fungal activity, anti-viral activity, weight control and thermogenesis, anti-diabetes, and anxiety reduction, mood enhancement and cognitive enhancement.

These embodiments of the disclosure, other embodiments, and their features and characteristics, will be apparent from the description, drawings and claims that follow.

### BRIEF DESCRIPTION OF THE DRAWINGS

FIG. 1 depicts an exemplary schematic diagram of supercritical carbon dioxide extraction of essential oil (Step 1) and decaffeination of green tea (Step 2) in accordance with the present invention.

FIG. 2 depicts an exemplary schematic diagram of ethanol extraction of crude green tea catechin chemical constituents fraction in accordance with the present invention.

FIG. 3 depicts an exemplary schematic diagram of an affinity adsorbent extraction process in accordance with the present invention.

FIG. 4 depicts an exemplary schematic diagram of water leaching extraction for L-theanine and polysaccharides in accordance with the present invention.

FIG. 5 depicts an exemplary schematic diagram of the purification of L-theanine and polysaccharide fractions in accordance with the present invention.

FIG. 6 depicts AccuTOF-DART Mass Spectrum for green tea polysaccharide fraction from step 6 of the present methods (positive ion mode).

FIG. 7 depicts AccuTOF-DART Mass Spectrum for green tea polysaccharide fraction from step 6 of the present methods (negative ion mode).

FIG. 8 depicts AccuTOF-DART Mass Spectrum for green tea polysaccharide fraction from step 6 of the present methods (positive ion mode).

FIG. 9 depicts AccuTOF-DART Mass Spectrum for green tea polysaccharide fraction from step 6 of the present methods (negative ion mode).

FIG. 10 depicts AccuTOF-DART Mass Spectrum for green tea polysaccharide fraction from step 6 of the present methods (positive ion mode).

FIG. 11 depicts AccuTOF-DART Mass Spectrum for green tea polysaccharide fraction from step 6 of the present methods (negative ion mode).

FIG. 12 depicts AccuTOF-DART Mass Spectrum for commercially available green tea (Kai Hua Long Ding) (positive ion mode).

FIG. 13 depicts AccuTOF-DART Mass Spectrum for green tea crude extract by 95% ethanol leaching from step 3 of the present methods (positive ion mode).

FIG. 14 depicts AccuTOF-DART Mass Spectrum for green tea phenolic acid feed from step 4 of the present methods by column chromatography using XAD 7HP desorption packing material (positive ion mode).

FIG. 15 depicts AccuTOF-DART Mass Spectrum for green tea purified F2 fraction from step 4 of the present methods by column chromatography using XAD 7HP desorption packing material (positive ion mode).

FIG. 16 depicts AccuTOF-DART Mass Spectrum for green tea purified F3 fraction from step 4 of the present methods by column chromatography using XAD 7HP desorption packing material (positive ion mode).

FIG. 17 depicts AccuTOF-DART Mass Spectrum for green tea purified F4 fraction from step 4 of the present methods by column chromatography using XAD 7HP desorption packing material (positive ion mode).

FIG. 18 depicts AccuTOF-DART Mass Spectrum for green tea purified F5 fraction from step 4 of the present methods by column chromatography using XAD 7HP desorption packing material (positive ion mode).

FIG. 19 depicts AccuTOF-DART Mass Spectrum for commercially available green tea (Kai Hua Long Ding) (negative ion mode).

FIG. 20 depicts AccuTOF-DART Mass Spectrum for green tea crude extract by 95% ethanol leaching from step 3 of the present methods (negative ion mode).

FIG. 21 depicts AccuTOF-DART Mass Spectrum for green tea phenolic acid feed from step 4 of the present methods by column chromatography using XAD 7HP desorption packing material (negative ion mode).

FIG. 22 depicts AccuTOF-DART Mass Spectrum for green tea purified F2 fraction from step 4 of the present methods by column chromatography using XAD 7HP desorption packing material (negative ion mode).

FIG. 23 depicts AccuTOF-DART Mass Spectrum for green tea purified F3 fraction from step 4 of the present methods by column chromatography using XAD 7HP desorption packing material (negative ion mode).

FIG. 24 depicts AccuTOF-DART Mass Spectrum for green tea purified F4 fraction from step 4 of the present methods by column chromatography using XAD 7HP desorption packing material (negative ion mode).

FIG. 25 depicts AccuTOF-DART Mass Spectrum for green tea purified F5 fraction from step 4 of the present methods by column chromatography using XAD 7HP desorption packing material (negative ion mode).

### DETAILED DESCRIPTION OF THE INVENTION

### Definitions

The articles “a” and “an” are used herein to refer to one or to more than one (i.e. to at least one) of the grammatical object of the article. By way of example, “an element” means one element or more than one element.

As used herein, “aerial parts” refers the constituent part of C. sinensis comprising leaves and stems.

As used herein, the term “catechin fraction” comprises the water soluble and ethanol soluble catechin compounds obtained or derived from green tea, further comprising, but not limited to, compounds such as ECGC, EGC, ECG, EC, GC, GCC, GC, and C.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

As used herein, the term “decaffeinated” comprises green extraction compositions that have a caffeine concentration less than that found in green tea leaf plant material.

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “essential oil fraction” comprises lipid soluble, water insoluble compounds obtained or derived from green tea including, but not limited to, the chemical compounds classified as n-hexadecanoic acid, tetradecanoic acid, 9-hexadecanol, E, oleyl alcohol, 1-octadecaol, phytol, and dihydroactinidiolide.

As used herein, “feedstock” generally refers to raw plant material, comprising whole plants alone, or in combination with on or more constituent parts of a plant comprising leaves, roots, including, but not limited to, main roots, tail roots, and fiber roots, stems, leaves, seeds, and flowers, wherein the plant or constituent parts may comprise material that is raw, dried, steamed, heated or otherwise subjected to physical processing to facilitate processing, which may further comprise material that is intact, cut, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “fraction” means the extraction composition comprising a specific group of chemical compounds characterized by certain physical, chemical properties or physical or chemical properties.

As used herein, term “green tea” refers to the leaves or aerial plant material derived from the Camellia sinensis species botanical. The term green tea is also used interchangeably with C. sinensis species and means these plants, clones, variants, and sports, etc. Green tea is the pharmaceutical name for conventional extraction products of the C. sinensis species plant material processed to produce green tea leaves.

As used herein, the term “green tea constituents’ shall mean chemical compounds found in green tea species and shall include all such chemical compounds identified above as well as other compounds found in green tea species, including but not limited to the essential oil chemical constituents, catechins, theanine, and polysaccharides.

As used herein, the term “one or more compounds” means that at least one compound, such as n-hexadecanoic acid (a lipid soluble essential oil chemical constituent of green tea), or ECGC (a water and water-ethanol soluble catechin of green tea), or theanine (a water soluble amino acid of green tea) or a water soluble-ethanol insoluble polysaccharide molecule of green tea is intended, or that more than one compound, for example, n-hexadecanoid acid and ECGC is intended. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents.

As used herein, the term “polysaccharide fraction” comprises water soluble-ethanol insoluble polysaccharide compounds obtained or derived from green tea. Non-limiting examples of polysaccharides include glucose, arabinose, galactose, rhamnose, xylose uronic acid and combinations thereof.

Other chemical constituents of green tea may also be present in these extraction fractions.

As used herein, the term “profile” refers to the ratios by percent mass weight of the chemical compounds within an extraction fraction or to the ratios of the percent mass weight of each of the four green tea fraction chemical constituents in a final green tea extraction composition.

As used herein, the term “purified” fraction or composition means a fraction or composition comprising a specific group of compounds characterized by certain physical-chemical properties or physical or chemical properties that are concentrated to greater than 50% of the fraction's or composition's chemical constituents. In other words, a purified fraction or composition comprises less than 50% chemical constituent compounds that are not characterized by certain desired physical-chemical properties or physical or chemical properties that define the fraction or composition.

The term “synergistic” is art recognized and refers to two or more components working together so that the total effect is greater than the sum of the components.

As used herein, the term “theanine fraction” comprises water soluble theanine, an amino acid obtained or derived from green tea.

The term “treating” is art-recognized and refers to curing as well as ameliorating at least one symptom of any condition or disorder.

Extractions

The present invention comprises extractions of isolated and purified fractions of essential oils, catechins, theanine, and polysaccharides from one or more green tea feedstocks. These individual fraction can be combined in specific ratios (profiles) to provide beneficial combinations and can provide extract products that are not found in currently known extract products. For example, an essential oil fraction from one species may be combined with a catechin fraction from the same or different species, and that combination may or may not be combined with a theanine fraction or polysaccharide fraction from the same or different green tea feedstock material. Such extractions include fractions that have predetermined amounts of at least one of the essential oil, catechin, theanine, or polysaccharide fractions. Embodiments comprise extractions of green tea that are free of oxalic acid. Embodiments comprise extractions of green tea that are decaffeinated.

Additional embodiments comprise extractions comprising altered profiles (ratio distribution) of the chemical constituents of the green tea in relation to that found in the native plant material or to currently available green tea extract products. For example, the essential oil fraction concentration may be increased or decreased in relation to the catechin and/or theanine and/or polysaccharide concentrations. Similarly, the catechins or theanine or polysaccharides may be increased or decreased in relation to the other extract constituent fractions to permit novel constituent chemical profile compositions for specific biological effects.

In one embodiment, an extraction of the present invention may comprise greater than 2% by mass weight of essential oil chemical constituents. Another embodiment of such extractions comprises a predetermined catechin concentration wherein the catechin concentration is greater than that found in the native plant material or conventional green tea species extracts. For example, an extraction may comprise novel green tea catechins at a concentration of greater than 30% by mass weight of the extraction. Another embodiment of such extractions may comprise an L-theanine concentration of greater than 2% by mass weight which is greater than the concentration of natural green tea L-theanine in the native plant material or currently available extraction products.

Alteration of the concentration relationships (chemical profiles) of the beneficial chemical constituents of the individual Green tea species permits the formulation of unique or novel Green tea species extraction products designed for specific human conditions or ailments. For example, a novel and powerful Green tea composition for anti-oxidant, oxygen free radical scavenging, and nitrosation inhibition activity could have a greater purified essential oil, catechin, and polysaccharide compositions and a reduced L-theanine composition by % mass weight than that found in the Green tea native plant material or conventional known extraction products. In contrast, a novel Green tea extraction for cancer prevention could have a greater purified essential oil and catechin fractions and reduced L-theanine and polysaccharide fractions by % mass weight than that found in the Green tea native plant material or conventional known extraction products. Another example of a novel Green tea extraction profile for anti-stroke and cerebral protection could be an extraction profile with a greater purified essential oil, catechin, L-theanine, and polysaccharide compositions by % mass weight than that found in native Green tea plant material or known conventional Green tea extraction products. For anti-aging activity, a high catechin fraction and reduced essential oil, theanine, and polysaccharide fractions by % mass weight than that found in native green tea plant material or conventional extraction products may be desirable. In contrast, for anxiety reduction, mood enhancement and cognitive enhancement, a greater purified theanine fraction and reduced essential oil, catechin, and polysaccharide fractions by % mass weight than that found in native green tea plant material or conventional extraction products may be the optimal composition product.

A further embodiment of the invention is extractions comprising novel sub-fractions of the catechin chemical constituents wherein the total catechins are highly purified (e.g., >95% by mass weight) and the concentration of specific highly bio-active catechin compounds such as ECGC has it's concentration increased relative to the other catechin compounds (profiled sub-fractions). Such novel and purified catechin sub-fraction extractions may be used alone or in combination with other green tea purified fractions, other botanical chemical constituents, or pharmaceutical chemical compounds. For example, such novel catechin sub-fractions may have substantial benefit for the prevention of cancer and aging.

Methods of the present invention comprise providing novel green tea extractions for treatment and prevention of human disorders. For example, a novel green tea extraction for antioxidant activity and cardiovascular protection may have an increased catechin fraction concentration, an increased essential oil fraction concentration, a decreased theanine concentration, and an increased polysaccharide fraction concentration, by % weight, than that found in the green tea native plant material or conventional known extraction products. A novel green tea species extraction for stroke prevention and therapy may have an increased catechin fraction, essential oil fraction, theanine fraction and a polysaccharide fraction concentration, by % weight, than that found in the native green tea plant material or conventional known extraction products. Another example of a novel green tea extraction for treatment of anxiety and depression comprises a composition having an increased theanine fraction concentration and a reduced essential oil fraction, and a reduced catechin concentration, and a reduced polysaccharide fraction than that found in native green tea plant material or known conventional extraction products.

Extractions Relative to Natural Green Tea

Embodiments comprise extractions of green tea having at least one of an essential oil, catechin, theanine, or polysaccharide concentration that is in an amount greater than that found in the native green tea plant material or currently available green tea extract products. Embodiments also comprise compositions wherein one or more of the fractions, including essential oils, catechins, theanine, or polysaccharides, are found in a concentration that is greater than that found in native green tea plant material. Embodiments also comprise extractions wherein one or more of the fractions, including essential oil, catechins, theanine, or polysaccharides, are found in a concentration that is less than that found in native green tea plant material. Known amounts of the bio-active chemical constituent fractions of green tea (Table 1) are used as an example of the present invention. For example, extractions of the present invention comprise fractions wherein the concentration of essential oils is from 0.001 to 200 times the concentration of native green tea plant material, and/or compositions wherein the concentration of catechins is from 0.001 to 4 times the concentration of native green tea plant material, and/or extractions wherein the concentration of theanine is from 0.001 to 200 times the concentration in green tea plant material, and/or extractions wherein the concentration of polysaccharides is from 0.001 to 40 times the concentration of native green tea plant material, and/or extractions wherein the concentration of caffeine is 0.001 to 0.99 times the concentration of green tea plant material. Extractions of the present invention comprise fractions wherein the concentration of essential oils is from 0.01 to 200 times the concentration of native green tea, and/or extractions wherein the concentration of catechins is from 0.01 to 4 times the concentration of native green tea, and/or extractions wherein the concentration of theanine is from 0.01 to 200 the concentration of native green tea, and/or extractions wherein the concentration of polysaccharides is from 0.01 to 40 times the concentration of native green tea plant material. Furthermore, extractions of the present invention comprise sub-fractions of the catechin chemical constituents having at least one or more of chemical compounds present in the native plant material catechin chemical constituents that is in an amount greater or lesser than that found in native green tea plant material catechin chemical constituents. For example, the chemical compound ECGC may have it's concentration increased in a catechin sub-fraction to 60% by % mass weight of the sub-fraction from it's concentration of 50% by % mass weight of the total catechin chemical constituents in the native green tea plant material. In contrast, C may have it's concentration reduced in a catechin sub-fraction to <0.1% by % mass weight of the sub-fraction from it's concentration of 2.2% by % mass weight of the total catechin chemical constituents in the native plant material. Extractions of the present invention comprise extractions wherein the concentration of specific chemical compounds in such novel catechin sub-fractions are either increase by about 1.1 to about 2 times or decreased by about. 0.1 to 100 times that concentration found in native green tea catechin chemical constituents.

A further embodiment of such extractions comprises a predetermined polysaccharide concentration substantially increased in relation to that found in natural Green tea species dried plant material or conventional Green tea species extract products. For example, an extraction may comprise the water-soluble ethanol insoluble polysaccharide fractions of greater than 3% of mass weight of the extraction. Embodiments also comprise extractions wherein one or more of the fractions, including the essential oil compounds, the catechins, L-theanine, or the polysaccharides, are found in a concentration that is less than that found in native Green tea plant material. For example, extractions of the present invention comprise the essential oils is from 0.001 to 100 times the concentration of native Green tea plant material, and/or extractions where the concentration of catechins is from 0.001 to 14 times the concentration of native Green tea plant material, and/or the concentration of L-theanine is from 0.001 to 100 times the native green tea plant material, and/or the polysaccharide concentration is from 0.001 to 80 times the concentration of native Green tea plant material. In making a combined extraction, from about 0.001 mg to about 200 mg of an essential oil fraction, can be used. Additionally, from about 0.001 mg to about 500 mg of a purified catechin fraction can be used. Furthermore, from about 0.001 mg to about 500 mg of a purified L-theanine fraction can be used. Finally, from about 0.001 mg to about 500 mg of the water-soluble, ethanol insoluble polysaccharide fraction can be used.

Purity of Extractions

The methods as taught in the present invention below permit the purification (concentration) of an essential oil fraction, a catechin fraction, catechin sub-fractions, a L-theanine fraction, and a polysaccharide fraction as well as decaffeination of the catechin, L-theanine, and polysaccharide fractions. An essential oil fraction purity as high as 89% by mass weight of the desired chemical constituents may be achieved with caffeine as the principal non-essential oil constituent in the purified fraction. SCCO2 has proven to be an excellent means for decaffeination of the green tea feedstock removing about 85% by mass weight of the caffeine in the feedstock material. Combining all of the sub-fractions of the process chromatography purification of the catechin fraction, a purity of total catechins of 63-68% by mass weight of the combined extract with a 57-69% ECGC concentration (profile) by mass weight of the total catechins may be obtained. Combining selected affinity adsorbent process chromatography elution sub-fractions, highly purified catechin sub-fractions comprising a total catechin purity of 91-99% by mass weight of the sub-fraction with a concentration of ECGC of 62-70% by mass weight of the total catechins is readily accomplish with a reasonably high yield. If yield is sacrificed, sub-fractions comprising even higher levels of total catechin purity and ECGC concentration may be obtained. A purified L-theanine fraction comprising an L-theanine concentration of 90% by mass weight of the fraction and a purified polysaccharide fraction comprising a polysaccharide concentration of greater than 90% by mass weight of the fraction with high yields are also accomplished using the methods as taught in the present invention. The specific extraction environments, rates of extraction, solvents, and extraction technology used depend on the starting chemical constituent profile of the source material and the level of purification desired in the final extraction products. Specific methods as taught in the present invention can be readily determined by those skilled in the art using no more than routine experimentation typical for adjusting a process to account for sample variations in the attributes of starting materials that is processed to an output material that has specific attributes. For example, in a particular lot of Green tea species plant material, the initial concentrations of the essential oil chemical constituents, caffeine, the catechins, L-theanine, and the polysaccharides are determined using methods known to those skilled in the art as taught in the present invention. One skilled in the art can determine the amount of change from the initial concentration of the catechin constituents, for instance, to the predetermined amounts of catechin chemical constituents for the final extraction product using the extraction methods, as disclosed herein, to reach the desired concentration in the final Green tea species composition product. Similarly, such changes can be made for the level of decaffeination and for the essential oil compounds, L-theanine, and polysaccharide fraction compositions.

In general, the methods and compositions of the present invention comprise methods for making an extracted Green tea species composition having predetermined characteristics. Such an extracted Green tea species composition may comprise any one, two, three, or all four of the four concentrated extract fractions depending on the beneficial biological effect(s) desired for the given product. Typically, a composition containing all four purified Green tea species extract fractions is generally desired as such novel compositions represent the first highly purified Green tea species extraction products that contain all four of the principal biologically beneficial chemical constituents found in the native plant material. Embodiments of the invention comprise methods wherein the predetermined characteristics comprise a predetermined selectively increased concentration of the Green tea species' essential oil compounds, catechins, L-theanine, and polysaccharides in separate extraction fractions. The importance of having all four of the biologically beneficial chemical constituent groups in final compositions is related to the synergistic interaction of these compounds in enhancing the desired physiological and medical effects of the green tea chemical constituents over that found with highly purified single chemical compounds or groups of related compounds.

Methods of Extraction

The starting material for extraction is plant material from one or more C. sinensis species. The plant material may be the any portion of the plant, though the aerial portion of the plant, which includes the leaves, stems, or other plant part is preferred. The leaves are the most preferred starting material.

The C. sinensis species plant material may undergo pre-extraction steps to render the material into any particular form, and any form that is useful for extraction is contemplated by the present invention. The C. sinensis leaf material is preferably steamed to inactivate the enzymes that convert the catechins to phlobphenes for the production of green tea. Such pre-extraction steps include, but are not limited to, that wherein the material is cut, chopped, minced, shredded, ground, pulverized, cut, or torn, and the starting material, prior to pre-extraction steps, is dried or fresh plant material. A preferred pre-extraction step comprises grinding and/or pulverizing the C. sinensis species leave material into a fine powder. The starting material or material after the pre-extraction steps can be dried or have moisture added to it. Once the green tea plant material is in a form for extraction, methods of extraction are contemplated by the present invention.

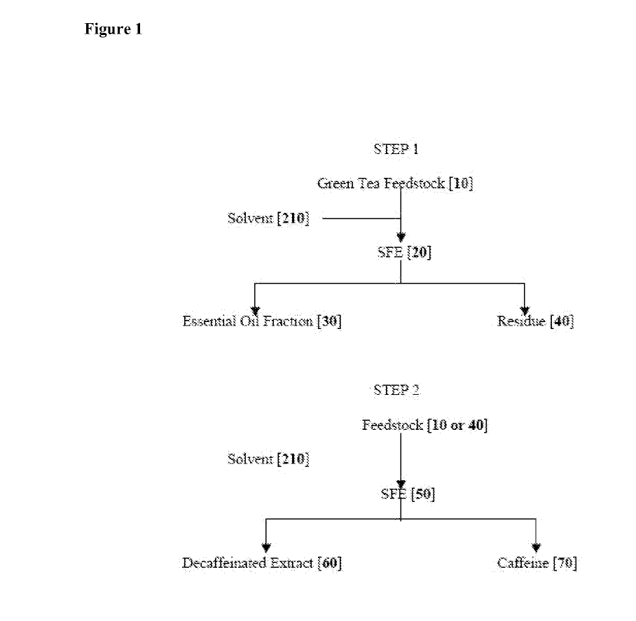

In general, methods of the present invention comprise, in part, methods wherein green tea plant material is extracted using supercritical fluid extraction (SFE), also termed supercritical carbon dioxide (SCCO₂), that is followed by one or more solvent extraction steps, such as, but not limited to, water, hydroalcoholic, and affinity polymer absorbent extraction processes. Additional other methods contemplated for the present invention comprise extraction of green tea plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, compositions of the present invention may be prepared by a method comprising the steps depicted schematically in FIGS. 1-5.

The invention includes methods for concentrating (purifying) and profiling the essential oil and other lipid soluble compounds from green tea plant material using SCCO2 technology. The invention includes the decaffeination of the green tea plant material using SCCO2 processing. Extraction of the essential oil chemical constituents and decaffeination of the green tea plant material with SCCO2 as taught in the present invention eliminates the use of toxic organic solvents. Carbon dioxide is a natural and safe biological product and an ingredient in many foods and beverages.

Essential oils are aromatic substances that are widely used in the perfume industries, in the pharmaceutical sector and in the food and human nutrition. They are mixture of more than 200 compounds, that can be grouped basically into two fractions, a volatile fraction, that constitutes 90-95% of the whole oil and contains monoterpenes and sesquiterpene hydrocarbon and their oxygenated derivatives, along with alphatic aldehydes, alcohols and esters, and a non-volatile residue, that constitutes from 5-10% of the whole oil and contains hydrocarbon, fatty acid, sterols, caroteroids, waxes, coumarins, psoraline and flavonoids.

The isolation, concentration and purification of essential oil have been important processes for many years, as a consequence of the widespread use of these compounds. The common methods used so far are mainly based on solvent extraction and steam distillation. The use of these conventional techniques has a major disadvantage (the risk of losses of thermolabile compounds) and also two significant drawbacks (the infeasibility for automation and the long time required for extraction). The commercial methods used for concentration are fractional vacuum distillation and selective solvent extraction and chromatographic separation. All these methods have important drawbacks, such as low yield, formation of byproducts (owing to the time of exposure to high temperature) and the presence of toxic organic residues in the extracts.

Supercritical fluid extraction (SFE) has been used recently for the extraction essential oils from plants in an attempt to avoid the drawbacks linked to conventional technique. Its usefulness for extraction is due to the combination of gas-like mass transfer properties and liquid-like solvating characteristics with diffusion coefficients greater than those of liquid solvents. SFE is also a suitable technique for enhancing the quality of essential oils obtained by conventional extraction methods by means of fractionation.

Caffeine, the most consumed alkaloid in the world, is found in high concentration in some natural products such as coca beans (0.2%), coffee beans (0.9-2.4%) and tea leaves (1.5-2.5%). Caffeine is commonly obtained by extraction using organic solvents, such as dichloromethane and hexane, which are considered harmful to human health and environment. Water is an excellent but a non-selective solvent for caffeine. Extraction with water leads to dissolution and subsequent loss of other valuable components such as the polyphenols (catechins) of green tea.

In the present invention, supercritical carbon dioxide has been chosen as the principal process for extract caffeine (decaffeination of green tea). This process involves using a compressed gas at high temperature as the solvent to remove caffeine. On a commercial scale, carbon dioxide is used to extract caffeine from coffee beans. Supercritical CO2 is non-polluting and nontoxic compared to the traditionally used organic solvents. Several patents have been issued for caffeine extraction from coffee beans with CO2 and have been previously discussed. Zosel (U.S. Pat. No. 4,247,570) detailed the operation of decaffeination on a commercial scale. The caffeine content in the coffee beans, ranging form 0.7 to 3%, was decreased to about 0.02% caffeine. The extraction process was conducted at 70-90 C and 160-200 bar (CO2 density of 0.4-0.65 g/cc).

Supercritical carbon dioxide is very selective for caffeine, but the solubility of caffeine is lower than in organic solvent, which results in the use of large quantities of CO2 and thereby a substantial increase in both fixed and operating costs. As observed with coffee beans, water can act as a valuable co-solvent leading to a substantially improved extraction yield.

A schematic diagram of the methods of extraction of the biologically active chemical constituents of green tea plant material is illustrated in FIGS. 1-5. The extraction process is typically, but not limited to, 6 steps. For reference in the text, when the bold number X appears in the text, the number refers to the number in FIGS. 1-5. The analytical methods used in the extraction process are presented in the Exemplification section.

Step 1: Supercritical Fluid Carbon Dioxide Extraction of Green Tea Essential Oil

Due to the hydrophobic nature of the essential oil, non-polar solvents, including, but not limited to SCCO₂, hexane, petroleum ether, and ethyl acetate may be used for this extraction process. Since some of the components of the essential oil are volatile, steam distillation may also be used as an extraction process.

A generalized description of the extraction of the essential oil chemical constituents from the leaves of green tea using SCCO2 is diagrammed in FIG. 1-Step 1. The feedstock [10] is dried cut green tea leaves (size greater than 105 μm). The extraction solvent [210] is pure carbon dioxide. Water may be used as a co-solvent. The feedstock is loaded into a into a SFE extraction vessel [20]. After purge and leak testing, the process comprises liquefied CO2 flowing from a storage vessel through a cooler to a CO2 pump. The CO2 is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO2 extractions taught herein are preferably performed at pressures of at least 100 bar and a temperature of at least 35° C., and more preferably at a pressure of about 60 bar to 300 bar and at a temperature of about 40° C. to about 60° C. The time for extraction for a single stage of extraction range from about 30 minutes to about 2.5 hours, to about 1 hour. The solvent to feed ratio is typically about 20-60 to 1 for each of the SCCO2 extractions. The CO2 is recycled for commercial extraction processing. The extracted, purified, and profiled essential oil chemical constituents [30] are then collected a collector or separator, saved in a light protective glass bottle, and stored in a dark refrigerator at 4° C. The Green tea feedstock [10] material may be extracted in a one step process (FIG. 1, Step 1A) wherein the resulting extracted and purified Green tea essential oil fraction [30] is collected in a one collector SFE or SCCO2 system [20]. Alternatively, as in a fractional SFE system, the SCCO2 extracted green tea feedstock material may be segregated into collector vessels (separators) such that within each collector there is a differing relative percentage essential oil chemical constituent composition (profile) in each of the purified essential oil sub-fractions collected. The residue (remainder) [40] is collected, saved and used for further processing to include, but not limited to, decaffeination and processing to obtain purified fractions of the green tea catechins, theanine, and polysaccharides. An embodiment of the invention comprises extracting the green tea feedstock material using multi-stage SCCO2 extraction at a pressure of 60 bar to 800 bar and at a temperature between 35° C. and 90° C. and collecting the extracted green tea material after each stage. A second embodiment of the invention comprises extracting the green tea species feedstock material using fractionation SCCO2 extraction at pressures of 60 bar to 800 bar and at a temperature between 35° C. and 90° C. and collecting the extracted green tea material in differing collector vessels at predetermined conditions (pressure, temperature, and density) and predetermined intervals (time). The resulting extracted green tea purified essential oil sub-fraction compositions from each of the multi-stage extractors or in differing collector vessels (fractional system) can be retrieved and used independently or can be combined to form one or more green tea essential oil compositions comprising a predetermined essential oil chemical constituent concentration that is higher or lower than that found in the native plant material. Typically, the total yield of the essential oil fraction from green tea plant material using a single step SCCO2 extraction is about 0.4% (>95% of the essential oil chemical constituents) by % weight having an essential oil chemical constituent purity of greater than 85% by mass weight of the extract. The results of such extraction processes are found below in Tables 2-4. The procedure can be found in Example 1.

TABLE 2
Results of 1^{st} stage processing at 40 C. and 200 bar.

| | | | | Caffeine | Caffeine |
|---|---|---|---|---|---|
| | | | Yield | purity | extracted from |
| Run | Feed¹ | S/F | (%) | (%) | feed (%)² |
| 2 | F1 dry leaf | 24 | 0.17 | 15.6 | 2.1 |
| 3 | F1 dry leaf | 24 | 0.13 | 18.3 | 1.9 |
| 4 | F1 dry leaf | 36 | 0.40 | 12.2 | 2.8 |
| 5 | F1 wet leaf with 40% | 24 | 0.26 | 21.6 | 4.6 |
| | water | | | | |
| 6 | F1 wet leaf with 100% | 60 | 0.45 | 79.8 | 17.5 |
| | water | | | | |
| 7 | F4 wet leaf with 100% | 60 | 0.66 | 11.0 | 2.2 |
| | water | | | | |
| 8 | JPGT wet leaf with | 60 | 0.88 | 16.8 | 2.1 |
| | 100% water | | | | |

¹F1 = Chinese green tea; F4 = Chinese green tea; JPGT = Japanese green tea.
²Caffeine extracted from feed = caffeine in extracts/caffeine in feed × 100.

TABLE 3
Composition of the essential oil extracts of Green tea.

| | Retention | | | | |
|---|---|---|---|---|---|
| Peak ID | time (min) | Name | CAS # | formula | Mw |
| CGTF2- | 7.8 | 4-Pentenal | 2100-17-6 | C5H8O | 84 |
| P1 | | | | | |
| CGTF2- | 11.5 | (Z)-2-octene | 7642-04-8 | C8H16 | 112 |
| P2 | | | | | |
| CGTF2- | 12.1 | 4-methylene-heptane | 15918-08-8 | C8H17 | 113 |
| P3 | | | | | |
| 1 | 13.5 | Heptanal | 111-71-7 | C7H14O | 114 |
| 2 | 16.0 | nonanal | 124-19-6 | C9H18O | 142 |
| CGTF2- | 18.3 | 2-propenoic acid-2- | 106-63-8 | C7H12O2 | 128 |
| P4 | | methylpropyl ester | | | |
| 3 | 20.0 | 2-methyl-1,3,4-oxadiazole | 3451-51-2 | C3H4N2O | 139 |
| 4 | 20.4 | 1,3-bis(1,1-dimethylethyl)- | 1014-60-4 | C14H22 | 190 |
| | | benzene | | | |
| 5 | 20.6 | 3-methyl-1-Heptanol | 1070-32-2 | C8H18O | 130 |
| 6 | 22.8 | tert-butyl acrylate | 1663-39-4 | C7H12O2 | 128 |
| 7 | 23.2 | 1-butoxy-pentane | 18636-66-3 | C9H20O | 144 |
| 8 | 23.6 | 4-ethyl-5-methyl-nonane | 1632-71-9 | C12H26 | 170 |
| 9 | 23.9 | unknown 1 | | C10H18O | 154 |
| JPGT- | 24.9 | 2-methoxy-1-(1-propenyl)- | 97-54-1 | C10H12O2 | 164 |
| P1 | | phenol | | | |
| 10 | 25.3 | 5-methyl-1-heptanol | 7212-53-5 | C8H18O | 130 |
| 11 | 26.1 | 3-butyl-cyclohexanone | 39178-69-3 | C10H18O | 154 |
| 12 | 28.2 | 2-methyl-2-nonanol | 10297-57-1 | C10H22O | 158 |
| JPGT- | 29.7 | 3-(bromomethyl)-Heptane | 18908-66-2 | C8H17Br | 192 |
| P2 | | | | | |
| 13 | 29.7 | 2,3,7-trimethyl octane | 62106-34-6 | C11H24 | 156 |
| 14 | 30.1 | 1-decanol | 112-30-1 | C10H22O | 158 |
| 15 | 31.5 | unknown 2 | | | 154 |
| JPGT- | 31.3 | 3-methyl-udecane | 1002-43-3 | C12H26 | 170 |
| P3 | | | | | |
| 16 | 31.7 | 3,5-bis(1,1-dimethylethyl)- | 1138-52-9 | C14H22O | 206 |
| | | phenol | | | |
| 17 | 32.4 | cyclohexanecarboxylic | 4840-76-0 | C9H14O2 | 154 |
| | | acid ethenyl ester | | | |
| 18 | 32.7 | methyl salicylate | 119-36-8 | C8H8O3 | 152 |
| 19 | 33.0 | Dodecane | 112-40-3 | C12H26 | 170 |
| CGTF2- | 33.1 | bicyclo(3,1,1)-heptan-3-ol | 27779-29-9 | C10H18O | 154 |
| P5 | | | | | |
| 20 | 33.4 | benzothiazole | 95-16-9 | C7H5NS | 135 |
| 21 | 34.9 | 2.2-dimethyl-undecane | 17312-64-0 | C13H28 | 184 |
| 22 | 35.3 | 3-methyl-undecane | 1001-43-3 | C12H26 | 170 |
| CGTF2- | 35.8 | 2,2-dimethyl-undecane | 17312-64-0 | C13H28 | 185 |
| P6 | | | | | |
| 23 | 36.6 | 2,6-dimethyl-2-octanol | 18479-57-7 | C10H22O | 158 |
| CGTF2- | 36.6 | 2-methyl-2-decanol | 2/9/3396 | C11H24O | 172 |
| P7 | | | | | |
| 24 | 37.9 | Indole | 120-72-9 | C8H7N | 117 |
| 25 | 38.3 | Tridecane | 629-50-5 | C13H28 | 184 |
| 26 | 38.9 | 1-undecanol | 112-42-5 | C11H24O | 172 |
| 27 | 39.3 | 3,7-dimethyl-nonane | 17302-32-8 | C11H24 | 156 |
| 28 | 39.7 | Butanoic acid, 3-hexenyl ester | 53998-84-8 | C10H18O2 | 170 |
| 29 | 40.4 | unknown 3 | | | |
| 30 | 41.5 | 4-Dodecanol | 10203-32-4 | C12H26O | 186 |
| 31 | 42.2 | 5-(2-methylpropyl)- | 62185-53-9 | C13H28 | 184 |
| | | Nonane | | | |
| 32 | 43.2 | Dodecanol | 112-54-9 | C12H24O | 184 |
| 33 | 43.8 | 3-(3,3-dimethylbutyl)- | 40564-98-5 | C12H24O | 184 |
| | | cyclohexanol | | | |
| 34 | 44.6 | 2-methyl-2-decanol | 3396-09-2 | C11H24O | 172 |
| 35 | 45.1 | Caffeine | 58-08-2 | C8H10N4O2 | 194 |
| 36 | 46.3 | n-butyl myristate | 110-36-1 | C18H36O2 | 284 |
| 37 | 47.0 | 1-Hexadecanol | 36653-82-4 | C16H34O | 242 |
| 38 | 48.2 | Nerolidol | | C15H26O | 222 |
| 39 | 49.4 | 1-Heptadecanol | 1454-85-9 | C17H36O | 256 |
| 40 | 49.8 | Hexadecanoic acid, methyl | 112-39-0 | C17H34O2 | 270 |
| | | ester | | | |
| 41 | 50.4 | unknown 4 | | | |
| 42 | 50.7 | unknown 5 | | | |
| 43 | 51.2 | unknown 6 | | | |
| 44 | 52.6 | n-hexadecanoic acid | 57-10-3 | C16H32O2 | 256 |
| 45 | 53.6 | unknown 7 | | | |
| 46 | 54.7 | 2-pentadecyl-1,3-dioxolane | 4360-57-0 | C18H36O2 | 284 |
| 47 | 55.3 | unknown 8 | | | |
| 48 | 56.1 | unknown 9 | | | |
| 49 | 56.2 | 5-cyclohexyl-dodecane | 13151-85-4 | C18H36 | 252 |
| 50 | 56.9 | unknown 10 | | | |
| 51 | 58.3 | unknown 11 | | | |
| 52 | 60.5 | Oleyl alcohol | 143-28-2 | C18H36O | 268 |
| 53 | 61.2 | (E)-9-octadecen-1-ol | 506-42-3 | C18H36O | 268 |
| 54 | 62.6 | unknown 12 | | | |
| 55 | 63.3 | Nonadecanol | 1454-84-8 | C19H40O | 284 |
| 56 | 64.5 | Nonadecane | | C19H42 | 268 |
| 57 | 65.2 | unknown 13 | | | |
| 58 | 65.5 | unknown 14 | | | |
| 59 | 66.8 | Phytol | 150-86-7 | C20H40O | 296 |
| 60 | 70.4 | oleic acid | 112-80-1 | C18H34O2 | 282 |
| 61 | 71.0 | unknown 15 | | | |
| 62 | 71.4 | 2-methyl-octadecane | 1560-88-9 | C19H40O | 268 |
| 63 | 74.3 | Octadecanoic acid | 57-11-4 | C18H36O2 | 284 |

TABLE 4
Green tea essential oil compounds distribution (Peak area %
from GC-MS) from different green tea feedstock.

| Peak ID | CGT F1 | CGT F2 | CGT F3 | CGT F4 | JPGT |
|---|---|---|---|---|---|
| CGTF2-P1 | | 0.18 | | | |
| CGTF2-P2 | | 0.11 | | | |
| CGTF2-P3 | | 0.17 | | | |
| 1 | | 0.28 | 0.19 | 0.22 | 0.16 |
| 2 | | 0.05 | 0.13 | 0.1 | |
| CGTF2-P4 | | 0.1 | | | |
| 3 | | | 0.08 | | |
| 4 | 0.1 | 0.14 | 0.24 | 0.21 | 0.13 |
| 5 | 0.23 | 0.8 | 0.1 | 0.05 | |
| 6 | 0.05 | 0.05 | 0.07 | 0.04 | 0.03 |
| 7 | 0.11 | 0.13 | 0.21 | 0.04 | 0.07 |
| 8 | 0.05 | | 0.1 | 0.06 | 0.05 |
| 9 | 0.12 | 0.21 | 0.29 | 0.24 | 0.05 |
| JPGT-P1 | | | | | 0.1 |
| 10 | | 0.05 | | 0.05 | |
| 11 | | 0.16 | 0.18 | 0.15 | 0.06 |
| 12 | | | 0.03 | 0.04 | 0.01 |
| JPGT-P2 | | | | | 0.04 |
| 13 | 0.04 | 0.07 | 0.07 | | |
| 14 | 0.12 | 0.23 | 0.34 | 0.15 | 0.19 |
| 15 | 0.06 | 0.21 | 0.27 | 0.21 | |
| JPGT-P3 | | | | | 0.04 |
| 16 | | | 0.08 | 0.06 | 0.08 |
| 17 | 0.47 | 0.06 | 0.14 | 0.07 | 0.11 |
| 18 | | | | 0.02 | |
| 19 | | | | 0.04 | 0.1 |
| CGTF2-P5 | | 0.13 | | | |
| 20 | | | | 0.04 | |
| 21 | | | | 0.03 | |
| 22 | | 0.04 | 0.06 | 0.05 | 0.05 |
| CGTF2-P6 | | 0.04 | | | |
| 23 | 0.06 | | 0.06 | 0.05 | 0.06 |
| CGTF2-P7 | | 0.09 | | | |
| 24 | | | | 0.03 | |
| 25 | 0.01 | 0.48 | 0.48 | 0.57 | 0.28 |
| 26 | 2.28 | 1.76 | 0.92 | 0.42 | 0.26 |
| 27 | 0.08 | 0.09 | 0.08 | 0.05 | 0.04 |
| 28 | | 0.03 | | 0.04 | 0.02 |
| 29 | | | 0.01 | | 0.04 |
| 30 | 0.59 | 0.04 | | 0.07 | 0.1 |
| 31 | | 0.12 | 0.51 | 0.08 | 0.17 |
| 32 | | | 0.06 | 0.06 | 0.1 |
| 33 | | 0.12 | | 0.18 | 0.08 |
| 34 | | | | 0.1 | 0.03 |
| 35 | 92.68 | 35.6 | 32.53 | 20.51 | 57.18 |
| 36 | | 0.36 | 0.84 | 0.21 | 0.35 |
| 37 | 0.2 | 19.03 | 15.46 | 21.56 | 10.95 |
| 38 | | 0.06 | 0.1 | 0.12 | |
| 39 | 0.27 | 0.18 | 1.15 | 0.04 | |
| 40 | | | 0.64 | 0.07 | |
| 41 | | 0.11 | | 0.07 | |
| 42 | | | | 0.06 | |
| 43 | | | | 0.04 | |
| 44 | 0.59 | 5.87 | 4.15 | 5.06 | 1.89 |
| 45 | | 0.11 | | 0.06 | |
| 46 | | 0.1 | 0.08 | 0.23 | 0.14 |
| 47 | 0.31 | 0.07 | 0.1 | 0.08 | 0.09 |
| 48 | | | | 0.05 | |
| 49 | | | 0.19 | | |
| 50 | | | | 0.11 | |
| 51 | | | | 0.1 | 1.65 |
| 52 | 0.12 | 9.44 | 13.76 | 22.56 | 10.29 |
| 53 | | 1.65 | 3.06 | 2.93 | 1.99 |
| 54 | | 0.06 | 0.53 | 0.1 | |
| 55 | 0.28 | 16.78 | 14.28 | 17.99 | 8.83 |
| 56 | | | 0.45 | | |
| 57 | | | 0.57 | | |
| 58 | 0.2 | 0.46 | 0.46 | 0.52 | 0.21 |
| 59 | 0.58 | 0.58 | 4.97 | 1.99 | 1.25 |
| 60 | | 0.36 | 0.73 | 0.66 | 0.23 |
| 61 | | | | 0.2 | |
| 62 | 0.11 | 1.98 | 0.22 | | 1.56 |
| 63 | | 0.98 | 0.87 | 0.92 | 0.53 |

A total of 73 compounds in green tea leaf essential oil fractions extracted using SCCO2 at 40° C. and 100-200 bar. It doesn't appear to matter whether the SCCO2 extraction is accomplished on either dry or wet green tea leaves, caffeine is extracted in this process. The caffeine concentration in these essential oil fractions varies from about 11-80% by % mass weight of the essential oil fraction. In addition to caffeine, other major compounds found in the essential oil fraction include saturated fatty alcohol such as 1-undecanol, 1-hexadecanol, oleyl alcohol, and nonadecanol and fatty acid such as hexadecanoic acid. Interestingly, very little in the way of essential oil chemical compounds were found in the Chinese green tea F1. In contrast, Chinese green tea F2, F3, and F4 all were found to have greater than 50% by mass weight fatty alcohols and fatty acids comprising the SCCO2 essential oil extraction fractions. In the SCCO2 essential oil fraction from Japanese green tea feedstock, less than 40% by mass weight fatty alcohols and fatty acids comprised the extract fraction.

Step 2. Supercritical Carbon Dioxide Decaffeination of Green Tea.

A generalized description of the decaffeination of the chemical constituents from the leaves of green tea using SCCO2 is diagrammed in FIG. 1-Step 2. The feedstock [10 or 40], dried cut green tea leaves (size greater than 105 μ) or the residue after the essential oil fraction extraction of Step 1, is soaked in one bed volume of distilled water The extraction solvent [210] is pure carbon dioxide. Water may be used as a co-solvent. The feedstock is loaded into a into a SFE extraction vessel [50]. After purge and leak testing, the process comprises liquefied CO2 flowing from a storage vessel through a cooler to a CO2 pump. The CO2 is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO2 extractions taught herein are preferably performed at pressures of at least 200 bar and a temperature of at least 35° C., and more preferably at a pressure of about 30 bar to 700 bar and at a temperature of about 60° C. to about 80° C. The time for extraction for a single stage of extraction range from about 2 to about 6 hours, to about 4 hour. The solvent to feed ratio is typically about 240 to 1 for each of the SCCO2 extractions. The CO2 is recycled for commercial extraction processing. The extracted caffeine chemical constituents [70] are then collected, measured for caffeine content, and discarded. The residue (remainder) or decaffeined green tea extract [60] is collected, saved and used for further processing to include, but not limited to, processing to obtain purified fractions of the green tea catechins, theanine, and polysaccharides. Typically, the total yield of the caffeine from green tea plant material using a single step SCCO2 extraction is about 4.5% (about 85% of the caffeine chemical constituents present in the feedstock) by % weight having a caffeine chemical constituent purity of about 29% by mass weight of the caffeine extract. Such a decaffeination process reduces the caffeine content in the decaffeinated green tea feedstock by about 55-85% by mass weight of the caffeine content in the feedstock material. In green tea feedstock having a low caffeine content, 83-85% mass weight of the caffeine can be removed. In order to reduce the caffeine content in high caffeine containing green tea feedstock, higher solvent/feed ratios are required to decaffeinate the feedstock material greater than 80% by mass weight. The results of such extraction processes are found below in Tables 5 and 6. The procedure can be found in Example 2.

TABLE 5
Results of total and caffeine extraction yield with
different co-solvent and feedstock.

| | | | | Caffeine |
|---|---|---|---|---|
| | | | Caffeine | extracted from |
| Cosolvent | S/F | Yield (%) | purity (%) | feed (%)¹ |
| | Dry tea leaf | | | |
| F1 N/A | 24 | 0.7 + 0.1² | 24.0 − 3.2 | 13.5 + 2.7 |
| F1 3% H2O | 60 | 1.1 | 15.9 | 13.4 |
| F1 4% 75% ethanol | 24 | 8.8 | 9.3 | 65.3 |
| F1 4% ethanol | 48 | 1.8 | 28.3 | 41.1 |
| | Wet leaf | | | |
| F1 wet with 40% water | 96 | 2.9 | 27.5 | 68.4 |
| F1 Wet with 100% water | 240 | 4.5 | 28.6 | 85.0 |
| F4 wet with 100% water | 240 | 3.1 | 41.0 | 55.4 |
| JPGT wet with 100% | 240 | 3.5 | 37.7 | 83.7 |
| water | | | | |

¹Caffeine extracted from feed = caffeine in extracts/caffeine in feed × 100.
²Results are averaged by three repeat runs (Standard deviation +/− 5%).

TABLE 6
Comparative HPLC analytical measurements of chemical constituents in decaffeinated
F1, F4, and JPGT green tea residues to the raw (natural) green tea feedstock, respectively.

| | | Yield of extracts (%) | | | | | | | | | Reduce |
|---|---|---|---|---|---|---|---|---|---|---|---|
| | | | | | | | | | | Total | caffeine |
| Feedstock | Feed | TheoB | EGC | C | CA | caff | EGCG | ECG | L-theanine* | PA | (%) |
| F1 | Raw | 0.02 | 2.51 | 0.15 | 0.10 | 1.25 | 3.57 | 0.68 | 0.73 | 6.92 | 85.7 |
| | Res | 0.02 | 2.26 | 0.21 | 0.12 | 0.18 | 3.87 | 0.76 | 0.69 | 7.10 | |
| F4 | Raw | 0.32 | 2.39 | 0.14 | 0.34 | 3.27 | 8.46 | 1.95 | 2.26 | 12.94 | 55.4 |
| | Res | 0.34 | 1.94 | 0.53 | 0.36 | 1.46 | 7.63 | 2.07 | 2.27 | 12.17 | |
| JPGT | Raw | 0.06 | 2.43 | 0.24 | 0.10 | 2.21 | 3.11 | 0.55 | 1.62 | 6.33 | 83.7 |
| | Res | 0.05 | 2.02 | 0.33 | 0.13 | 0.36 | 3.22 | 0.66 | 1.61 | 6.23 | |

*L-theanine yield was tested in water

The extraction yield of caffeine increased with the addition of co-solvent. The solubility of caffeine in supercritical fluid carbon dioxide/co-solvent mixture is 3-5 times higher than that in pure carbon dioxide alone (Kopcak 2005). From the standpoint of caffeine extraction, 75% ethanol/water is very efficient. However, in addition to 9.3% by mass weight of caffeine in the decaffeination extract, valuable phenolic acid chemical compounds from the feedstock are also extracted such as 4% EGCG, 2.6% EGC, and 0.9% ECG by % mass weight of the decaffeination extract. Water is a better co-solvent than ethanol for the decaffeination of the green tea leaf feedstock material. Using wet green tea leaves, water as a co-solvent, and a S/F ratio of 240, greater than 80% of the caffeine in the F1 feedstock and the Japanese green tea (JPGC) feedstock can be removed (decaffeinated) without removing any of the valuable phenolic acids or theanine from the feedstock material. This equates to a reduction of the caffeine content from 1.3% mass weight in the F1 feedstock to 0.18% in the F1 decaffeinated material or a reduction of caffeine content 2.2% by mass weight in the JPGT feedstock to 0.36% in the decaffeinated green tea material, a 6-7 fold decrease in caffeine content. In the case of the high caffeine content 3.3% by mass weight F4 green tea leaf feedstock, the reduction of total caffeine was less. 1.46% mass weight of the F4 decaffeinated feedstock. However, the valuable catechins and theanine were preserved in the decaffeinated residue material. Thus, the decaffeinated residue that retains the valuable catechin and theanine chemical constituents can then be used for further processing to obtain purified catechin, theanine, and polysaccharide fractions.

Interestingly, only about 55% decaffeination was observed for the high caffeine content F4 feedstock under the same SFE decaffeination conditions. The difference appears to be caused by either the higher caffeine content or a different matrix structure of the F4 green tea leaves or both. Based on observation, F4 feedstock leaves is wetted less by water soaking. In other words, the water tends to remain on the surface of the leaves instead of penetrating into the F4 leave internal matrix. Therefore, F4 green tea leaves will require more water and/or longer soaking times to achieve greater than 80% decaffeination as well as possibly a greater solvent/feed ratio.

Step 3. Ethanol Extraction of Crude Green Tea Catechin Chemical Constituents Fraction.

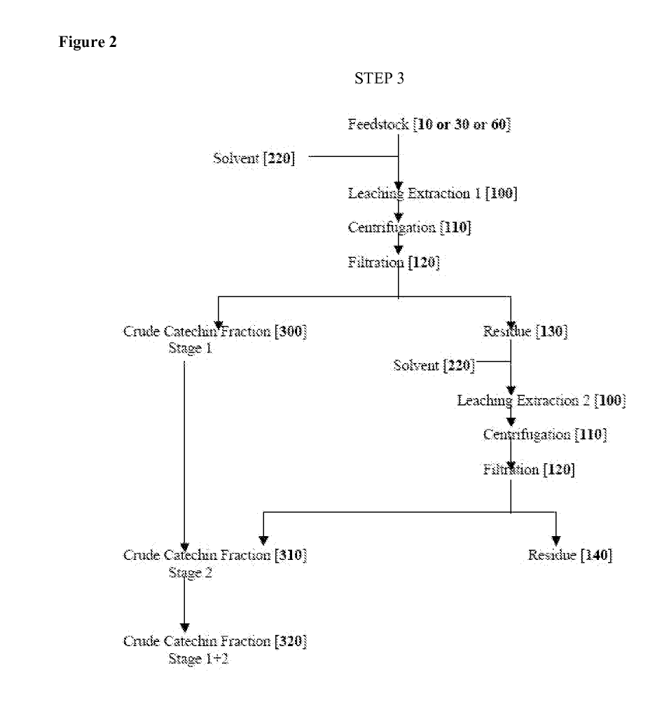

In one aspect, the present invention comprises extraction and concentration of the bio-active catechin chemical constituents. A generalized description of this step is diagrammed in FIG. 2-Step 3. This Step 3 extraction process is a solvent leaching process. The feedstock for this extraction is either tea cut green tea leaf material Green tea [10] or the residue from either the Step 1 SCCO2 extraction the essential oil fraction [30] or the Step 2 SCCO2 decaffeination of the green tea leaf material [60]. The extraction solvent 220 is 95% ethanol. The extraction solvent may be 10-95% aqueous alcohol, 95% aqueous ethanol is preferred. In this method, the green tea feedstock material and the extraction solvent are loaded into an extraction vessel 100 that is heated and stirred. It may be heated to 90° C., to about 80° C., to about 70° C., or to about 60-90° C. The extraction is carried out for about 1-10 hours, for about 1-4 hours, for about 2 hours. The resultant fluid extract is centrifuged [110] and filtered [120]. The filtrate (supernatant) [300, 310] is collected as product, measured for volume and solid content dry mass after evaporation of the solvent. The extraction residue material [130 or 140 is retained and saved for further processing (see Step 4). The extraction may be repeated as many times as is necessary or desired. It may be repeated 2 or more times, 3 or more times, 4 or more times, etc. When more that one stage is used for extraction, the crude catechin fractions from each stage may be combined [320] for product or retained for further purification of the catechin fraction (see Step 4). For example, FIG. 2-Step 3 shows a two stage process, wherein the second stage uses the same methods and conditions. The results are presented in Tables 7 and 8 below. The procedure can be found in Example 3.

TABLE 7
Results of 95% ethanol 2 stage leaching extraction of F1 green tea decaffeination residue

| | | Purity in extracts (%) | | | | | | | | |
|---|---|---|---|---|---|---|---|---|---|---|
| | Yield | | | | | | | | | Total |
| Solvent | (%) | TheoB | EGC | C | CA | caff | EGCG | ECG | L-theanine | PA* |
| EtOH | 23.2 | 0.15 | 14.39 | 0.77 | 0.63 | 1.09 | 15.34 | 3.27 | 0.19 | 33.77 |
| 95% | | | | | | | | | | |

| | Yield of extracts (%) | | | | | | | | |
|---|---|---|---|---|---|---|---|---|---|
| Solvent | TheoB | EGC | C | CA | caff | EGCG | ECG | L-theanine | Total PA |
| EtOH | 0.03 | 3.36 | 0.18 | 0.15 | 0.26 | 3.58 | 0.76 | 0.04 | 7.88 |
| 95% | | | | | | | | | |

*PA (phenolic acids) = EGC + C + EGCG + ECG

TABLE 8
Chemical constituents content comparisons of 95% ethanol 2 stage leaching extraction
products from native (raw) green tea feedstock and SFE decaffeinated residue for
Chinese green tea F1, Chinese green tea F4, and Japanese green tea (JPGT).

| | Yield | Purity in extracts (%) | | | | | | | |
|---|---|---|---|---|---|---|---|---|---|
| Sample | (%) | TheoB | EGC | C | CA | caff | EGCG | ECG | Total PA |
| FI - raw leaf | 27.5 | 0.09 | 9.13 | 0.54 | 0.36 | 4.53 | 12.98 | 2.48 | 25.14 |
| F1 - SFE | 19.3 | 0.11 | 11.66 | 1.11 | 0.62 | 0.92 | 20.02 | 3.92 | 36.71 |
| residue | | | | | | | | | |
| F4 - raw leaf | 36.7 | 0.9 | 6.51 | 0.39 | 0.93 | 8.93 | 23.07 | 5.33 | 35.30 |
| F4 - SFE | 31.1 | 1.09 | 6.25 | 1.69 | 1.14 | 4.69 | 24.54 | 6.66 | 39.15 |
| residue | | | | | | | | | |
| JPGT - raw leaf | 23.6 | 0.2 | 10.3 | 1.0 | 0.4 | 9.4 | 13.2 | 2.3 | 26.81 |
| JPGT - SFE | 24.7 | 0.2 | 8.2 | 1.3 | 0.5 | 1.5 | 13.1 | 2.7 | 25.26 |
| residue | | | | | | | | | |

| | Yield of extracts (%) | | | | | | | |
|---|---|---|---|---|---|---|---|---|
| Sample | TheoB | EGC | C | CA | caff | EGCG | ECG | Total PA* |
| FI - raw leaf | 0.02 | 2.51 | 0.15 | 0.10 | 1.25 | 3.57 | 0.68 | 6.92 |
| F1 - SFE residue | 0.02 | 2.26 | 0.21 | 0.12 | 0.18 | 3.87 | 0.76 | 7.10 |
| F4 - raw leaf | 0.32 | 2.39 | 0.14 | 0.34 | 3.27 | 8.46 | 1.95 | 12.94 |
| F4 - SFE residue | 0.34 | 1.94 | 0.53 | 0.36 | 1.46 | 7.63 | 2.07 | 12.17 |
| JPGT - raw leaf | 0.06 | 2.43 | 0.24 | 0.10 | 2.21 | 3.11 | 0.55 | 6.33 |
| JPGT - SFE residue | 0.05 | 2.02 | 0.33 | 0.13 | 0.36 | 3.22 | 0.66 | 6.23 |

*PA = total catechins (EGC + C + EGCG + ECG).

These results demonstrate that the SFE decaffeination process removed the caffeine from the green tea feedstock without affecting the other valuable chemical compounds in the residue. Furthermore, extraction of the residue using 95% ethanol preserves the L-theanine and water soluble-ethanol insoluble polysaccharides in the residue that may be used for further processing for purified theanine and polysaccharide fractions. Finally, the two stage leaching process increases the concentration of the four principal catechins (PA, Table 8) from about 7-12% by mass weight in the native green leaf feedstock to about 26-39% by mass weight in the extract, an about 3.5 fold increase in purity. The extraction yield ranged from 19 to 36% by mass weight based on the original green tea feedstock. Additional purity of the catechin chemical constituent fraction may be obtained using an affinity adsorbent process chromatography processes (see below).

Step 4. Affinity Adsorbent Extraction Process

As taught herein, a highly purified catechin fraction extract from green tea may be obtained by contacting a hydroalcoholic extract of green tea feedstock (Step 3) with a solid affinity polymer adsorbent resin so as to adsorb the active catechins contained in the hydroalcoholic extract onto the affinity adsorbent. The bound chemical constituents are subsequently eluted by the methods taught herein. Prior to eluting the catechin fraction chemical constituents, the affinity adsorbent with the desired chemical constituents adsorbed thereon may be separated from the remainder of the extract in any convenient manner, preferably, the process of contacting with the adsorbent and the separation is effected by passing the aqueous extract through an extraction column or bed of the adsorbent material. Moreover, prior to eluting the catechin fraction chemical constituents, any caffeine compounds adsorbed onto the affinity adsorbent may be separated from the catechins by using a specific solvent that will elute the caffeine compounds but not elute the catechin compounds (decaffeination of the purified catechin fractions).

A variety of affinity adsorbents can be utilized to purify the catechin chemical constituents of green tea plant material, such as, but not limited to “Amberlite XAD-2” (Rohm & Hass), “Duolite S-30” (Diamond Alkai Co.), “SP207” (Mitsubishi Chemical), ADS-5 (Nankai University, Tianjin, China), ADS-17 (Nankai University, Tianjin, China), Dialon HP 20 (Mitsubishi, Japan), and Amberlite XAD7 HP (Rohm & Hass). Amaberlite XAD 7HP is preferably used due to the high affinity for the catechin chemical constituents of green tea. It is a nonionic alphatic acrylic polymer with particle size of 560-710 μn that derives its adsorptive properties from its macroreticular structure (containing both a continuous polymer phase and a continuous pore phase), high surface area, and aliphatic nature of its surface. With this macroreticular structure having polymeric ester groups, XAD 7HP can adsorb polar compounds yielding a high affinity for phenolic acids (catechins).

Although various eluants may be employed to recover the catechin chemical constituents from the adsorbent, in one aspect of the present invention, the eluant comprises low molecular weight alcohols, including, but not limited to, methanol, ethanol, or propanol. In a second aspect, the eluant comprises low molecular alcohol in an admixture with water. In a third aspect, the eluant comprises low molecular weight alcohol, a second organic solvent, and water. In another aspect, an eluant used for decaffeinating the catechins adsorbed onto the absorbent comprises an acidic solvent such as, but not limited to, 5% H2SO4 in 10% ethanol. Thus, a two-stage elution process has been designed for purification of the catechin chemical constituent fraction of green tea. The first stage is to use an acidic solution to decaffeinate the chemical constituents adsorbed on the column by taking advantage of the base property of caffeine and the acid property of the catechins. The second stage is to use an ethanol/water eluant to elute the decaffeination catechins.

The green tea feedstock may or may not have undergone one or more preliminary purification processes such as, but not limited to, the processes described in Step 1, 2 and 3 prior to contacting the aqueous catechin chemical constituent containing extract with the affinity adsorbent material.

Using affinity adsorbent processes as taught in the present invention results in highly purified, profiled, and decaffeinated catechin chemical constituent fractions of the green tea that are remarkably free of other chemical constituents which are normally present in natural plant material or in available commercial extraction products. For example, the processes taught in the present invention can result in purified catechin extracts that contain total catechin chemical constituents in excess of 95% by dry mass weight.

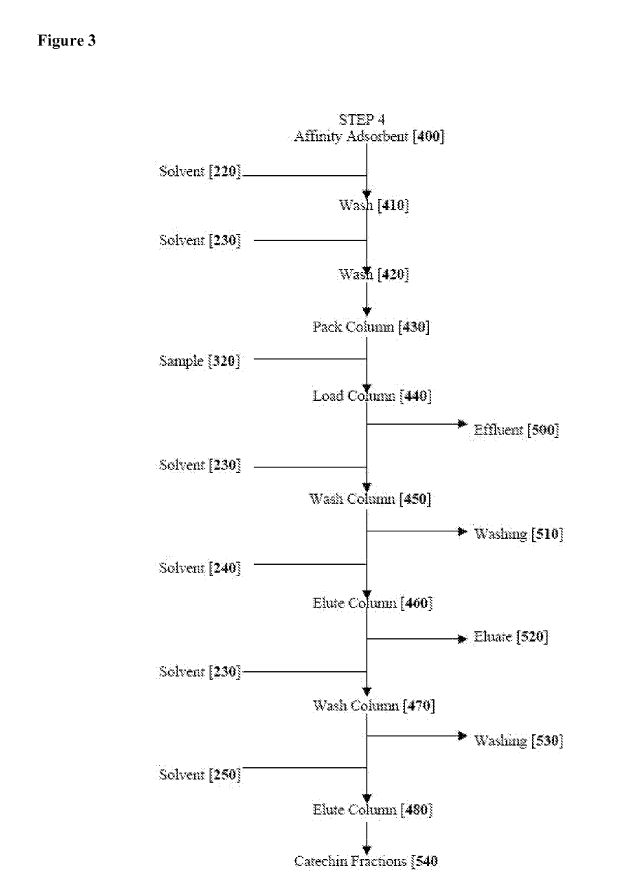

A generalized description of the extraction and purification of the catechins from the leaves of the green tea using polymer affinity adsorbent resin beads is diagrammed in FIG. 3-Step 4. The feedstock for this extraction process may be either the natural green tea feedstock [10] or the aqueous solution containing the catechins from Step 3 95% Ethanol Leaching Extraction [320]. The appropriate weight of adsorbent resin beads (12 mg of catechins per gm of adsorbent resin) is washed with 4-5 BV ethanol [220] and 4-5 BV distilled water [230] before and after being loaded into a column 410, 420. The cleaned adsorbent resin beads are packed into a column [430]. The catechin containing aqueous solution [320] is then loaded onto the column [440] at a flow rate of 2 to 4 bed volume (BV)/hour. Once the column is fully loaded, the column is washed [450] with distilled water [230] at a flow rate of 2-3 BV/hour to remove any impurities from the adsorbed catechins. The effluent residue [500] and washing residue [510] were collected, measured for mass content, catechin content, caffeine content, and discarded. Elution of the adsorbed caffeine compounds [460] is accomplished in an isocratic fashion with 5% H2SO4 in 10% ethanol as an eluting solution [240] at a flow rate of 2-4 BV/hour. The eluate [520] is collected, measured for mass content, catechin content, caffeine content, and discarded. After this decaffeination stage, the column is washed [470] with 8 BV of distilled water [230] at a flow rate of 10 BV/hour. The washing [530] is tested by pH paper until it is neutral, collected, and discarded. Elution of the adsorbed catechins [480] is accomplished in an isocratic fashion with 80% ethanol/water solution as an elution solution [250] at a flow rate of 2-4 BV/hour and the elution curve was recorded for the eluate extract [540]. Elution volumes 480 may be collected about every 15-30 minutes and these samples are analyzed using HPLC and tested for solids content and purity. The results are presented in Tables 9-11. The procedure can be found in Example 4.

TABLE 9
Analytical results of XAD 7HP column process chromatography of F1 SFE
decaffeinated 95% ethanol leaching green tea extract.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 23.3 | 480 | 181.16 | 91.50 | 66.42 | 20.06 | 3.19 | 0.68 | 6.20 | 1.85 |
| Effluent | | 170 | | | | | | | 1.22 | |
| F2 | 3.89 | 38.2 | 51.63 | 29.58 | 16.35 | 5.08 | 0.62 | | 0.16 | 0.69 |
| F3 | 1.84 | 55 | 26.15 | 14.11 | 8.50 | 2.91 | 0.63 | | 0.08 | 0.59 |
| F4 | 2.65 | 35.3 | 37.39 | 21.35 | 11.08 | 4.36 | 0.59 | | 0.15 | 0.42 |
| F5 | 1.70 | 10.5 | 31.56 | 19.58 | 7.32 | 4.32 | 0.34 | | 0.08 | 0.41 |
| F6 | 0.51 | 0.9 | 10.22 | 6.46 | 1.83 | 1.93 | 0.00 | | 0.00 | 0.00 |
| Total | 10.6 | 390 | 157.0 | 91.1 | 45.1 | 18.6 | 2.2 | | 0.5 | 2.1 |
| Recovery | | 81.5 | 86.6 | 99.5 | 67.9 | 92.7 | 68.4 | | 7.6 | 114.2 |
| (%) | | | | | | | | | | |

| | | | Total | | | | | | | |
|---|---|---|---|---|---|---|---|---|---|---|
| | | Yield | TPs | EGCG | EGC | ECG | C | TheoB | Caff | CA |
| Sample | Collect | (%) | (%) | (%) | (%) | (%) | (%) | (%) | (%) | (%) |
| Loading | | 23.3 | 37.4 | 18.9 | 13.7 | 3.9 | 4.1 | 0.14 | 1.3 | 0.38 |
| F2 | 0.8-1 BV | 3.89 | 64.0 | 36.7 | 20.3 | 6.3 | 0.8 | | 0.20 | 0.86 |
| F3 | 1-1.1 BV | 1.84 | 68.5 | 37.0 | 22.3 | 7.6 | 1.6 | | 0.21 | 1.55 |
| F4 | 1.1-1.3 BV | 2.65 | 68.0 | 38.8 | 20.1 | 7.9 | 1.1 | | 0.3 | 0.8 |
| F5 | 1.3-1.6 BV | 1.70 | 89.5 | 55.6 | 20.8 | 12.2 | 1.0 | | 0.2 | 1.15 |
| F6 | 1.6-3 BV | 0.51 | 97.2 | 61.4 | 17.4 | 18.3 | | | | |
| F2-F4 | 0.8-1.3 V | 8.38 | 66.2 | 37.41 | 20.67 | 7.10 | 0.98 | | 0.22 | 0.98 |
| F5-F6 | 1.3-3 V | 2.21 | 91.3 | 56.90 | 20.00 | 13.64 | 0.89 | | 0.17 | 0.89 |

TABLE 10
Analytical results of XAD-7HP column process chromatography of F4 SFE
decaffeinated 95% ethanol leaching green tea extract.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 26.6 | 2250 | 1096.7 | 521.28 | 339.5 | 136.0 | 100 | 40 | 100 | 37 |
| Effluent | | 540 | | | | | | | | |
| F2 | 0.4 | 35 | 21.3 | 13.8 | 4.4 | 1.8 | 1.3 | | 1.4 | 1.1 |
| F3 | 9.2 | 779 | 0.0 | 377.4 | 67.6 | 89.7 | 15.8 | | 26.7 | 15.4 |
| F4 | 4.1 | 347 | 213.7 | 152.0 | 16.3 | 39.8 | 5.5 | | 8.9 | 5.1 |
| F5 | 1.0 | 85 | 57.6 | 39.8 | 2.7 | 13.8 | 1.4 | | 1.3 | 1.5 |
| F6 | 0.2 | 14 | 292.6 | 583.1 | 91.1 | 145.1 | 23.9 | | 38.3 | 23.2 |
| total | 14.9 | 1261 | 585.2 | 1166.2 | 182.2 | 290.1 | 47.9 | | 76.7 | 46.3 |
| Recovery | | 56.1 | 53.4 | — | 53.7 | — | 47.9 | | 76.1 | — |
| (%) | | | | | | | | | | |

| | | Yield | Total TPs | EGCG | EGC | ECG | C | TheoB | Caff | CA |
|---|---|---|---|---|---|---|---|---|---|---|
| Sample | Collect | (%) | (%) | (%) | (%) | (%) | (%) | (%) | (%) | (%) |
| Loading | | 26.6 | 48.8 | 23.2 | 15.1 | 6.1 | 4.4 | 1.8 | 4.5 | 1.7 |
| F2 | 0.8-1 | 0.4 | 60.7 | 39.4 | 12.6 | 5.0 | 3.6 | 0.25 | 4.0 | 3.3 |
| | BV | | | | | | | | | |
| F3 | 1-1.6 | 9.2 | 70.7 | 48.4 | 8.7 | 11.5 | 2.0 | | 3.4 | 2.0 |
| | BV | | | | | | | | | |
| F4 | 1.6-2.3 | 4.1 | 61.5 | 43.8 | 4.7 | 11.5 | 1.6 | | 2.6 | 1.5 |
| | BV | | | | | | | | | |
| F5 | 2.3-2.9 | 1.0 | 67.9 | 46.9 | 3.2 | 16.2 | 1.6 | | 1.5 | 1.7 |
| | BV | | | | | | | | | |
| F6 | 2.9-3.7 | 0.2 | 98.9 | 66.6 | 3.2 | 29.1 | 0.0 | | 2.5 | 0.0 |
| | BV | | | | | | | | | |
| F2-F5 | 0.8-2.9 V | 14.8 | 67.7 | 46.8 | 7.3 | 11.6 | 1.9 | | 3.1 | 1.9 |

TABLE 11
Analytical results of XAD-7HP column process chromatography of
JPGT SFE decaffeinated 95% ethanol leaching green tea extract.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 24.2 | 1910 | 542 | 285.85 | 174.30 | 59.48 | 22.46 | 4.35 | 33.22 | 10.54 |
| Effluent | | 800 | | | | | | | 1.22 | |
| F2 | 5.3 | 420 | 273.1 | 177.4 | 49.3 | 38.4 | 8.0 | | 3.7 | 3.9 |
| F3 | 2.3 | 182 | 112.2 | 78.2 | 13.1 | 18.0 | 2.9 | | 1.0 | 1.4 |
| F4 | 0.3 | 23.5 | 19.6 | 14.1 | 1.4 | 3.5 | 0.6 | | 0.1 | 0.2 |
| F5 | 0.1 | 6.5 | 3.0 | 2.7 | | 1.1 | | | | |
| total | 8.0 | 631.5 | 407.9 | 272.1 | 63.8 | 60.5 | 11.5 | | 4.8 | 5.5 |
| Recovery | | 33 | 75.2 | 95.2 | 36.6 | 101.8 | 51.2 | | 14.4 | 51.9 |
| (%) | | | | | | | | | | |

| | | | Total | | | | | | | |
|---|---|---|---|---|---|---|---|---|---|---|
| | | Yield | TPs | EGCG | EGC | ECG | C | TheoB | Caff | CA |
| Sample | Collect | (%) | (%) | (%) | (%) | (%) | (%) | (%) | (%) | (%) |
| Loading | | 24.2 | 28.4 | 15.0 | 9.2 | 3.1 | 1.2 | 0.23 | 1.7 | 0.6 |
| F2 | 0.7-1.2 BV | 5.3 | 65.1 | 42.3 | 11.7 | 9.1 | 1.9 | | 0.9 | 0.9 |
| F3 | 1.2-2.0 BV | 2.3 | 61.7 | 43.0 | 7.2 | 9.9 | 1.6 | | 0.6 | 0.8 |
| F4 | 2.0-2.7 BV | 0.3 | 83.5 | 60.0 | 6.0 | 15.0 | 2.4 | | 0.4 | 0.9 |
| F5 | 2.7-3.6 BV | 0.1 | 98.0 | 69.1 | | 28.9 | | | | |

An acidic elution solvent has proven to be an excellent process for further decaffeination of the catechins reducing the concentration of caffeine in the final products to less than 1% to as low as 0.2% by mass weight of the extract. A purified fraction of catechins can be obtained with purity of >90% with a total yield of 1.9% by % mass weight of the original green tea feedstock. Furthermore, a sub-fraction may also be obtained wherein the concentration of EGCG is increased to >60% with a catechin purity of >95% irrespective of the original green tea feedstock used. A summary of the catechin purity and ECGC profile in the combined process chromatography eluates of F1, F4, and JPGT is shown in Table 12.

TABLE 12
Comparison of catechin purity and EGCG profile* in combined
(F2-F5 or F6) process chromatography eluates derived from
two-stage 95% ethanol leaching of three different
SFE decaffeinated green tea feedstock.

| | PA feed | | Combined fractions | | Combined fractions | |
|---|---|---|---|---|---|---|
| | Purity | EGCG profile | | EGCG profile | | EGCG profile |
| | (%) | (%) | Purity (%) | (%) | Purity (%) | (%) |
| F1 | 37.4 | 51 | 66.2 (1.8 times) | 57 | 91.3 (2.4 times) | 62 |
| F4 | 48.8 | 47 | 67.7 (1.4 times) | 69 | 98.9 (2 times) | 67 |
| JPGT | 28.4 | 53 | 63 (2.2 times) | 67 | 98 (2.9 times) | 70 |

*Profile = % mass weight of the four principal catechins

In other typical experiments, the working solution was the transparent aqueous solution obtained after Step 3 95% leaching extraction of raw or original green tea leaf feedstock material. For these experiments, 25 gm raw Green tea residue was leaching extracted using 250 ml of 95% ethanol at 70° C. two stages with 2 hours in each stage (solvent/feed ratio of 20/1). The two supernatant solutions from this two-stage extraction were combined and ethanol extraction solvent was removed using a rotary evaporator. After removing ethanol (distillation), some solid precipitation occurred that was removed using centrifugation and filtration as described in Step 3. The supernatant was collected and then distilled water was added to the concentrated supernatant to achieve a final concentration of 16-30 mg/ml. This transparent aqueous solution containing the catechins was then used for further purification using the affinity adsorbent process chromatography methods of Step 4. The results of this Step 3 leaching extraction are found in Table 13 wherein the leaching of raw or original green tea leaves are compared to the leaching of SFE decaffeinated residue from Step 2. It should be noted that in the case of the raw green tea plant material, a precipitation occurred during the ethanol distillation that was not observed with the SFE decaffeinated residue. Therefore, the centrifugation and filtration of this precipitate reduced the total yield from 32.8% to 26.9% by % mass weight based on the original feedstock material. Although the total yield is higher from the leaching extracted raw green tea feedstock, the purity of the catechin chemical constituents is similar. Furthermore, the caffeine concentration is very high in the raw green tea leaching extraction product. The results of such two-stage 95% ethanol leaching are tabulated in Tables 13 and 14.

TABLE 13
Comparison of yield and purity of Step 3 95% ethanol leaching extracts of F1 raw
green tea leaf (Raw) to F1 SFE decaffeinated green tea residue of Step 2 (Res).

| | | | Purity in Extracts (%)* | | | | | | | | |
|---|---|---|---|---|---|---|---|---|---|---|---|
| | | Yield | | | | | | | | | Total |
| Solvent | Feed | (%) | TheoB | EGC | C | CA | Caff | EGCG | ECG | L-theanine | Cat** |
| 95% | Raw | 32.8 | 0.18 | 10.86 | 0.46 | 0.51 | 5.52 | 16.91 | 3.19 | 0.12 | 31.43 |
| Ethanol | Res | 23.2 | 0.15 | 14.39 | 0.77 | 0.63 | 1.09 | 15.34 | 3.27 | 0.19 | 33.77 |
| Post | Raw | 26.9 | 0.17 | 15.67 | 0.89 | 0.65 | 7.42 | 22.67 | 4.21 | 0.13 | 43.44 |
| Distillation | | | | | | | | | | | |

| | | Yield of extracts (%) | | | | | | | | |
|---|---|---|---|---|---|---|---|---|---|---|
| | | | | | | | | | | Total |
| Solvent | Feed | TheoB | EGC | C | CA | Caff | EGCG | ECG | L-theanine | Cat |
| 95% | Raw | 0.06 | 3.57 | 0.15 | 0.17 | 1.81 | 5.55 | 1.05 | 0.04 | 10.32 |
| Ethanol | Res | 0.03 | 3.36 | 0.18 | 0.15 | 0.26 | 3.58 | 0.76 | 0.04 | 7.88 |
| Run 2 Post | Raw | 0.05 | 4.21 | 0.24 | 0.18 | 1.99 | 6.09 | 1.13 | 0.04 | 11.67 |
| Distillation | | | | | | | | | | |

*Purity defined as the concentration (% dry mass weight of the compound in the extract).
**TheoB-theobromine; CA-chlorogenic acid; Caff-caffeine; Cat-catechins.

TABLE 14
Yield and purity of two-stage 95% ethanol leaching extracts of F1, F4, and JPGT raw
green tea leaf feedstock.

| | | Purity in extracts (%) | | | | | | | |
|---|---|---|---|---|---|---|---|---|---|
| Feed | Yield (%) | TheoB | EGC | C | CA | caff | EGCG | ECG | Total PA |
| F1 | 26.9 | 0.17 | 15.67 | 0.89 | 0.65 | 7.42 | 22.67 | 4.21 | 43.44 |
| F4 | 37.7 | 1.3 | 8.4 | 2.2 | 1.2 | 12.5 | 32.4 | 7.8 | 50.76 |
| JPGT | 24.6 | 0.27 | 9.46 | 1.06 | 0.43 | 12.99 | 21.42 | 4.02 | 35.97 |

| | Yield of extracts (%) | | | | | | | |
|---|---|---|---|---|---|---|---|---|
| Feed | TheoB | EGC | C | CA | caff | EGCG | ECG | Total PA |
| F1 | 0.05 | 4.21 | 0.24 | 0.18 | 1.99 | 6.09 | 1.13 | 11.67 |
| F4 | 0.48 | 3.16 | 0.84 | 0.44 | 4.72 | 12.22 | 2.94 | 19.16 |
| JPGT | 0.08 | 2.76 | 0.31 | 0.13 | 3.79 | 6.24 | 1.17 | 10.48 |

The typical adsorption experiments were carried out at room temperature in an open batch system. ˜30 g PA XAD7HP were washed with ethanol to remove monomers and impurities and then soaked in distilled water for 16 hours before packing. Then, the clean PA resin beads were packed into a 10 mm (ID)×350 mm (L) glass column. 100 ml aqueous solution (de-ethanolized leaching solution) having a concentration of 16-30 mg/ml was loaded into the packed column at flow rate of 1.8 ml/min, 2 BV/hr. Following loading, 150 ml of distilled water was used to wash the column at flow rate of 10 BV/hr. Then, 200 ml of 5% sulfuric acid in 10% ethanol was used to elute (de-caffeinate) the column at a flow rate of 2.2 BV/hr. Following this elution, 250 ml of distilled water was used to wash the column at 10 BV/hr until the washings from column reached a pH of 7. Then, 100 ml of 80% ethanol was used to elute (de-adsorption) the column at a flow rate of 2 BV/hr. The total processing time was 300 min. Sequential eluant fractions were collected. Each eluate fraction was assayed by HPLC and the results are shown in Tables 15-17.

TABLE 15
Yield and purity of extract fractions of F1 green tea raw leaf as feedstock for Step 3
leaching followed by Step 4 process chromatography.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 26.9 | 1630 | 707.57 | 369.19 | 255.25 | 68.61 | 14.51 | 2.74 | 120.85 | 10.64 |
| Effluent | | 340 | | | | | | | 0.56 | |
| F2 | 1.1 | 66.08 | 48.79 | 34.38 | 9.25 | 4.36 | 0.80 | | 0.12 | 1.61 |
| F3 | 4.7 | 285.25 | 235.70 | 160.82 | 42.62 | 26.59 | 5.67 | | 1.61 | 2.83 |
| F4 | 3.7 | 226.48 | 165.45 | 125.36 | 11.70 | 26.19 | 2.19 | | 0.28 | 2.16 |
| F5 | 1.7 | 105.36 | 67.39 | 49.85 | 2.45 | 14.54 | 0.55 | | 0.13 | 0.99 |
| F2-F5 | | 683.2 | 517.33 | 370.41 | 66.02 | 71.68 | 9.22 | | 2.14 | 7.59 |
| Recovery | | 41.9 | 73.1 | 100 | 25.8 | 100 | 63.5 | | 1.8 | 71.3 |
| (%)* | | | | | | | | | | |

| | | | Total | | | | | | | |
|---|---|---|---|---|---|---|---|---|---|---|
| | | Yield | TPs | EGCG | EGC | ECG | C | TheoB | | CA |
| Sample | Collect | (%) | (%) | (%) | (%) | (%) | (%) | (%) | Caff (%) | (%) |
| Loading | | | 43.3 | 22.7 | 15.7 | 4.2 | 0.9 | 0.2 | 7.41 | 0.65 |
| F1 | −0.5 BV | | | | | | | | | |
| F2 | 0.5-1.0 BV | 1.1 | 73.8 | 52.0 | 14.0 | 6.6 | 1.2 | | 0.19 | 2.43 |
| F3 | 1.0-1.5 BV | 4.7 | 82.6 | 56.4 | 14.9 | 9.3 | 2.0 | | 0.56 | 0.99 |
| F4 | 1.5-2.0 BV | 3.7 | 73.1 | 55.4 | 5.2 | 11.6 | 1.0 | | 0.12 | 0.95 |
| F5 | 2.0-3 BV | 1.7 | 64.0 | 47.3 | 2.3 | 13.8 | 0.5 | | 0.13 | 0.94 |
| F2-F5 | 0.5-3 BV | 11.3 | 75.7 | 54.2 | 9.7 | 10.5 | 1.3 | | 0.31 | 1.1 |

*recovery was calculated by: weight of (F2-F5)/loading × 100

TABLE 16
Yield and purity of extract fractions of F4 green tea raw leaf F4 as feedstock for Step
3 leaching followed by Step 4 process chromatography.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 37.7 | 3040 | 1543.9 | 984.7 | 254.3 | 237.3 | 67.6 | 38.69 | 380.7 | 35.6 |
| Effluent | | 420 | | | | | | | 0.56 | |
| F2 | 6.0 | 487.3 | 391.5 | 266.4 | 39.9 | 74.8 | 10.3 | | 13.2 | 9.3 |
| F3 | 4.1 | 333.0 | 310.6 | 204.3 | 39.8 | 58.0 | 8.5 | | 11.2 | 6.9 |
| F4 | 1.0 | 77.1 | 68.0 | 45.8 | 4.3 | 15.6 | 2.2 | | 1.7 | 1.4 |
| Total | 11.2 | 899 | 771.2 | 517.4 | 84.1 | 148.8 | 21.0 | | 26.1 | 17.6 |
| Recovery (%)* | | 29.5 | 50.0 | 52.5 | 33.0 | 62.7 | 31.0 | | 6.9 | 49.5 |

| | | | Total | | | | | | | |
|---|---|---|---|---|---|---|---|---|---|---|
| | | Yield | TPs | EGCG | EGC | ECG | C | TheoB | Caff | CA |
| Sample | Collect | (%) | (%) | (%) | (%) | (%) | (%) | (%) | (%) | (%) |
| Loading | | | 50.8 | 32.4 | 8.36 | 7.8 | 2.2 | 1.3 | 12.5 | 1.2 |
| F1 | −0.8 BV | | | | | | | | | |
| F2 | 0.8-1.1 BV | 6.0 | 80.4 | 54.7 | 8.2 | 15.4 | 2.1 | | 2.7 | 1.9 |
| F3 | 1.1-1.8 BV | 4.1 | 93.3 | 61.3 | 11.9 | 17.4 | 2.5 | | 3.4 | 2.1 |
| F4 | 1.8-3.0 BV | 1.0 | 88.1 | 59.5 | 5.6 | 20.2 | 2.8 | | 2.2 | 1.8 |
| F2-F4 | 0.8-3.0 BV | 11.2 | 85.8 | 57.6 | 9.4 | 16.5 | 2.9 | | 2.9 | 2.0 |

*recovery was calculated by: weight of (F2-F4)/loading × 100

TABLE 17
Yield and purity of extract fractions of Japanese green tea raw leaf as feedstock for
Step 3 leaching followed by Step 4 process chromatography.

| | | Total | Total | | | | | | | |
|---|---|---|---|---|---|---|---|---|---|---|
| | Yield | solids | TPs | EGCG | EGC | ECG | C | TheoB | Caff | CA |
| Sample | (%) | (mg) | (mg) | (mg) | (mg) | (mg) | (mg) | (mg) | (mg) | (mg) |
| Loading | 29.1 | 2460 | 884.3 | 526.6 | 232.7 | 98.9 | 26.1 | 6.70 | 319.4 | 10.7 |
| Effluent | | 940 | | | | | | | | |
| F2 | 5.5 | 465.1 | 333.72 | 209.8 | 71.0 | 43.4 | 9.56 | | 5.07 | 2.66 |
| F3 | 2.2 | 186 | 115.74 | 83.2 | 13.1 | 17.4 | 2.01 | | 1.83 | 1.47 |
| F4 | 0.5 | 45.4 | 25.25 | 19.1 | 1.9 | 4.2 | 0.00 | | 0.18 | 0.66 |
| Total | 8.3 | 899 | 474.7 | 312.1 | 86.1 | 65.0 | 11.6 | | 7.1 | 4.8 |
| Recovery (%)* | | 28.3 | 53.7 | 59.3 | 37.0 | 65.7 | 44.3 | | 2.2 | 44.9 |

| | | | Total | | | | | | | |
|---|---|---|---|---|---|---|---|---|---|---|
| | | Yield | TPs | EGCG | EGC | ECG | C | TheoB | Caff | CA |
| Sample | Collect | (%) | (%) | (%) | (%) | (%) | (%) | (%) | (%) | (%) |
| Loading | | | 36.0 | 21.4 | 9.46 | 4.0 | 1.0 | 0.3 | 13.0 | 0.4 |
| F1 | −0.6 BV | | | | | | | | | |
| F2 | 0.6-1.2 BV | 6.0 | 71.8 | 45.1 | 15.3 | 9.3 | 2.1 | | 1.09 | 0.6 |
| F3 | 1.2-2.1 BV | 4.1 | 62.2 | 44.8 | 7.0 | 9.4 | 1.1 | | 0.98 | 0.8 |
| F4 | 2.1-3.0 BV | 1.0 | 55.6 | 42.1 | 4.3 | 9.3 | | | 0.41 | 1.5 |
| F2-F4 | 0.6-3.0 BV | 11.2 | 68.2 | 44.8 | 12.4 | 9.3 | 1.7 | | 1.0 | 0.7 |

*recovery was calculated by: weight of (F2-F4)/loading × 100

In the Step 4 affinity adsorbent purification of the catechins starting with raw (un-decaffeinated) green tea plant material, 95% of the caffeine can be removed using an acidic elution solvent while preserving the bond of the catechins to the adsorbent. For example, it is possible to decaffeinate crude leaching extracts containing about 12% caffeine by % mass weight to 0.3% in a process chromatography extract fraction. The higher the concentration of caffeine in the feedstock, the larger the volume of acid solution eluant is required to decaffeinate the extraction fractions.

Interestingly, elution of the catechins using 80% ethanol results in a greater yield of the EGCG, ECG and C than EGC by % mass weight in the extract fractions. The highest yields and purity are found in the 0.6 to 2 BV fractions. The total catechin purity highly purified fractions was about 1.7 times that of the crude leaching extracts. The level of catechin purity was not as high as that achieved with Step 4 process chromatography of SFE decaffeinated residue. However, total extract fraction yields of greater than 11% mass weight based on the original green tea feedstock are achieved with total catechin purities of 71% to 93% depending on the green tea feedstock. Finally the chemical distribution profile of the four principal catechins are altered with a preferential increase in the % mass weight of EGCG, ECG, and C and a decrease in EGC. For example, EGCG is typically greater than 65% by mass weight of the total catechins in the combined extract fractions and may be as high as 75% by mass weight in the extract sub-fractions.

Upon analysis for oxalic acid in the purified catechin fractions and sub-fractions, no oxalic acid could be detected in any of these fractions or sub-fractions despite the presence of oxalic acid in the 95% ethanol leaching feedstock. In fact oxalic acid was found to be as high as 6% by mass weight in the feedstock solutions. However, the oxalic acid compounds did not adsorb onto the affinity adsorbent and were found in the effluent.

Step 5. Water Leaching for Theanine and Polysaccharides.

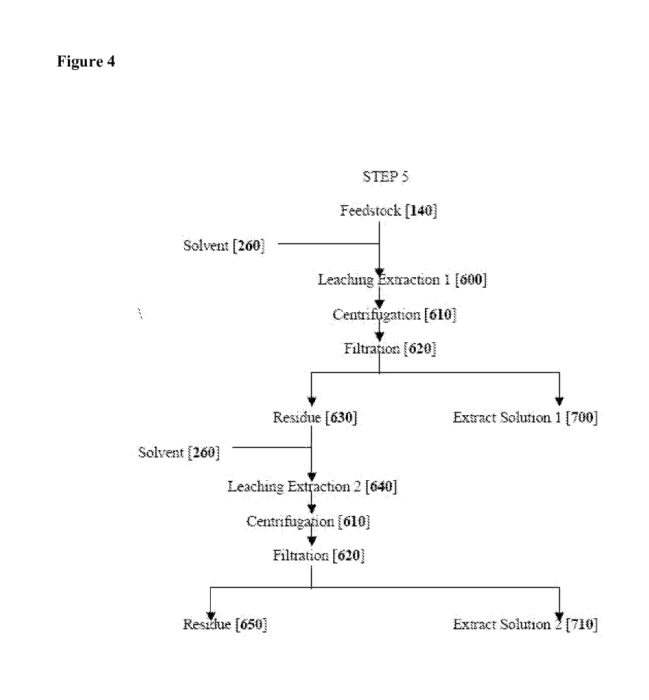

The polysaccharide extract fraction of the chemical constituents of green tea has been defined in the scientific literature as the “water soluble, ethanol insoluble extraction fraction”. Both L-theanine and the polysaccharides are soluble in water. A generalized description of the extraction of theanine and the polysaccharides from extracts of green tea plant material using water solvent leaching is diagrammed in FIG. 4-Step 5 (Appendix 1). The feedstock 140 is the solid residue from the 95% leaching extraction process of Step 3. This feedstock is leaching extracted in two stages. The solvent is distilled water 260. In this method, the green tea extract residue 140 and the extraction solvent 260 are loaded into an extraction vessel 600 and heated and stirred. It may be heated to 100° C., to about 80° C., or to about 60-80° C. The extraction is carried out for about 1-hours, for about 2-4 hours, or for about 2 hours. The supernatant extract solutions 700 are centrifuged 610, filtered 620 and collected. The residue 630 is retained and saved for further processing. The extraction may be repeated on the residue as many times as is necessary or desired. It may be 2 or more times, 3 or more times, 4 or more times, etc. For examples FIG. 4-Step 5 shows a two stage process, where the second stage uses the same methods and conditions. The final residue [650] discarded. An example of this extraction step is found in Example 5 and the results of mass measurement and HPLC analysis for L-theanine content are shown in Table 18.

TABLE 18
Water leaching, polysaccharide purification, and theanine purification results
obtained from different green tea feedstock.

| Steps | | CGT F1 | | | | CGT F4 | | | JPGT | |
|---|---|---|---|---|---|---|---|---|---|---|
| Water leaching | Water leaching | 3.6 | | | | 12.5 | | | 10.7 | |
| | yield (%) | | | | | | | | | |
| | L-theanine purity | 16.7 | | | | 18.2 | | | 13.2% | |
| | (%) | | | | | | | | | |
| | L-theanine yield | 0.59 | | | | 2.27 | | | 1.41 | |
| | (%) | | | | | | | | | |
| | Extracted L- | 88.0 | | | | 93.4 | | | 88.6 | |
| | theanine from | | | | | | | | | |
| | feed (%) | | | | | | | | | |
| 75% Et-OH | Pcp yield (%) | 1.15 | | | | 5.2 | | | 8.5 | |
| precipitation | Polysaccharide | 5K | 50K | 410K | 5K | 50K | 410K | 5K | 50K | 410K |
| | purity by dextran | 38 | 33 | 27 | 50 | 42 | 36 | 34 | 28 | 23.2 |
| | (%) | | | | | | | | | |

| | | | | |
|---|---|---|---|---|
| | Supernatant yield | 1.5 | 4.5 | 3.16 |
| | (%) | | | |
| | Theanine purity | 31.1 | 42.1 | 38 |
| | in supernatant | | | |
| | (%) | | | |
| Re- | Yield (%) | 0.51 | 1.95 | 1.24 |
| crystallization | L-theanine purity | 90 | 92.3 | 91 |
| | (%) | | | |

| | | | |
|---|---|---|---|
| Recovered L-theanine (%) | 68.5 | 74.0 | 69.7 |

The total yield of the water leaching process was from 3.6-12.5% by mass weight of the original green tea feedstock material. The concentration of L-theanine was 13.2-18.2% by mass weight of the leaching extract. Greater than 85% yield by mass weight of the theanine in the original green tea leaf feedstock may be extracted with the two-stage leaching process. Consistent with the scientific literature (29), the other chemical constituents should largely be the polysaccharides. An additional Step 6 may be used for separation of the theanine from the polysaccharide chemical constituents.

Step 6. Purification of L-Theanine and Polysaccharide Fractions.

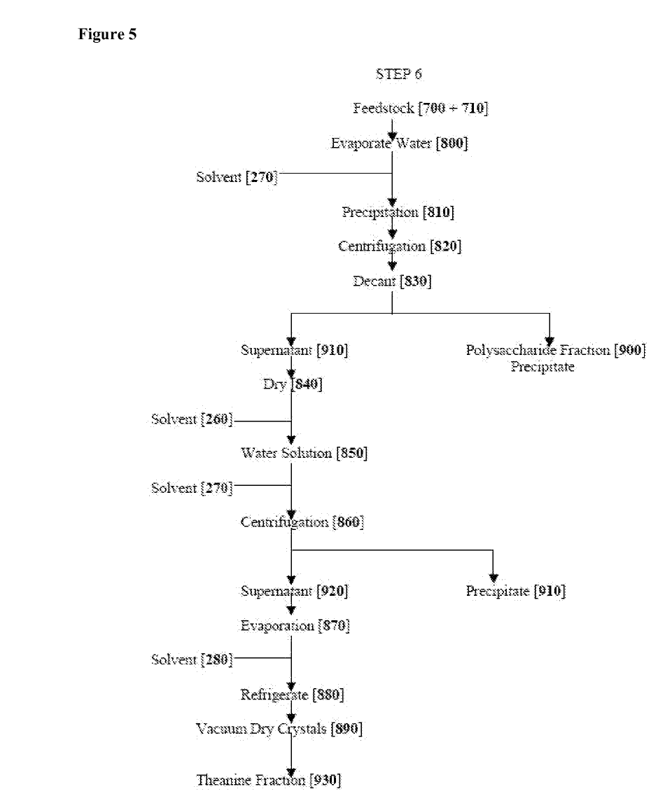

A generalized description of the extraction and purification of the polysaccharide and theanine fractions from extracts of green tea using water solvent processes is diagrammed in FIG. 5-Step 6. The feedstock is the water leaching supernatant solutions [700+710] from Step 5 water leaching extraction. The combined solutions are evaporated [800] to remove 60% of the water. The solvent absolute ethanol [280] is then added to the concentrated solution to make a final ethanol concentration at 75%. The solution is allowed to stand and a large precipitate [810] is observed. The solution is centrifuged [820], decanted [830] and the supernatant [910] is collected for further processing and purification of the theanine fraction. The precipitate product [900] is the purified polysaccharide fraction that may be analyzed for polysaccharides using the colormetric method by using Dextran 5,000-410,000 molecular weight as reference standards. The purity of the extracted polysaccharide fraction is about 23-50% based on different molecular weights of dextran with a total yield of 1.15% by % mass weight of the original native green tea leaf feedstock (Table 18). Combining the various dextran equivalent purities is consistent with an overall polysaccharide purity of greater than 90% by mass weight of the purified polysaccharide fraction.

The theanine purity in the supernatant solution is about 31-42%. To achieve a higher level of theanine purity, additional processing is required. The supernatant solution [910] is dried. The dried product is dissolved in sufficient distilled water [260] to make a 10% solution [850]. To this solution, 4 volumes of absolute ethanol [270] is added and mixed. This hydroalcoholic solution is allowed to sit for about 1 hour and then centrifuged [860] and any precipitate [910] is discarded. The supernatant [920] is concentrated using vacuum rotary evaporator [870] at about 60° C. to achieve an 80% solution. This 80% solution is allowed to cool to room temperature and then 4 volumes of absolute ethanol [280] are added to the solution which is refrigerated [880] at 0° C. for 24 hours. The crystals that formed are collected and vacuum dried [890] at 60° C. to yield the purified theanine fraction [930]. The purified theanine fraction yield is about 0.51-1.95% by % mass weight of the original green tea feedstock with a purity of about 90-92% (Table 18). The actual procedure can be found in Example 6.

The green tea polysaccharide yield was 1.2-8.5% by mass weight based on the original green tea leaf feedstock. The purity of the polysaccharide fraction was 23-50% based on different molecular weights of dextran indicating an overall purity of >90% green tea polysaccharide chemical constituents in the fraction. Based on a large number and variety of experimental approaches, it is quite reasonable to conclude that 1.2-8.5% yield by mass weight is greater than 90% of the water soluble, ethanol insoluble polysaccharides in the natural green tea species feedstock material.

The green tea L-theanine yield was 0.5-2.0% by mass weight based on the original green tea feedstock which about 70% of the theanine in the original feedstock. A theanine purity of 90% can be achieved using these methods.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions wherein alcohol was previously removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

In performing the previously described extraction methods, it was found that greater than 90% yield by mass weight of the essential oil chemical constituents having greater than 80% purity of the essential oil chemical constituents present in the original dried leaf feedstock of Green tea and related species can be extracted in the essential oil SCCO2 extract fraction (Step 1). Using the methods as taught in Step 2 (SCCO2 Decaffeination Processes), the total yield of the caffeine from green tea plant material is about 4.5% by mass weight (about 85% of the caffeine compounds present in the original green tea feedstock) having a caffeine chemical purity of about 29% by mass weight of the caffeine extract. Such a decaffeination process reduces the caffeine content in the decaffeinated green tea feedstock to below 0.2% by % mass weight of the decaffeinated green tea material. Moreover, using the methods as taught in the present invention, 80% decaffeination of the green tea feedstock material may be achieved while maintaining the valuable catechin, theanine, and polysaccharide chemical constituents in the feedstock which can be used for further processing to obtain purified catechin, theanine, and polysaccharide fractions.

Using the methods as taught in Step 3 of this invention, an ethanol leaching fraction is achieved with a 19-31% yield by mass weight from the original Green tea species feedstock. The yield of the catechin chemical constituents is greater than 90% by mass weight of the catechins present in the original green tea feedstock (see Tables 8 and A1-Appendix 1). Moreover, the ethanol leaching process increases the concentration (purity) of the four principal catechins from 7-12% by mass weight in the native green tea leaf feedstock's studied to about 25-39% by mass weight in the catechin extract fraction, a 3.5 fold increase in the concentration of catechins (sum of ECGC, ECG, EGC, and C). Finally, the ethanol leaching extraction preserves the theanine and polysaccharide chemical compounds in the solid residue that may be used for further processing for purified theanine and polysaccharide fractions (Steps 5 & 6).

Using the methods as taught in Step 4 of this invention (Affinity Adsorbent Extraction Processes), catechin fractions with purities of greater than 90% by % dry mass of the extraction fraction may be obtained. It is possible to extract 56-86% of the catechins from the 95% ethanol leaching extract feedstock. This equates to a 50-77% yield of the catechin chemical constituents found in the native Green tea species plant material using ECGC, ECG, EGC and C as the catechin chemical constituent references. Based on HPLC analysis of this phenolic acid fraction using these as references, the purity of the phenolic acid chemical constituents is about 40% of the phenolic acid fraction extraction products. In addition, an acidic elution solvent has proven to be an excellent process for further decaffeination of the purified catechin fraction reducing the caffeine in the final catechin fraction products to less than 1% to as low as 0.2% by mass weight of the extract fraction. Furthermore, sub-fractions may be obtained wherein the concentration of EGCG is increased to 65-75% by mass weight with a catechin purity of greater than 95% by mass weight of the extract sub-fraction The data supports the ability of the affinity adsorbent process chromatography to profile the catechin extract fractions by preferentially increasing the % mass weight of EGCG, ECG, and C and decreasing EGC in the extract fraction.

Using the methods as taught in Step 5 (Water Leaching Process), a high yield of water-soluble chemical constituents of about 3.6% by mass weight based on the original green tea leaf feedstock. The concentration of L-theanine in this crude extract is about 17% by mass weight. The remaining water-soluble compounds are largely polysaccharides which is consistent with the results of other studies (29) wherein they reported that the concentration of polysaccharide in green tea leaves was about 2.42% by mass weight with a purity of 86.8% using 60% ethanol precipitation.

Using the methods as taught in Step 6 of this invention (L-theanine and Polysaccharide Extraction and Purification Processes), the total yield of water-soluble ethanol-insoluble polysaccharides is about 1.2% by mass weight based on the original feedstock. The purity of the polysaccharide extract fraction is about 56-76% based on a colormetric method using different molecular weights of dextran as reference standards. These data are consistent with a total polysaccharide purity of greater than 95%.

Moreover, using the methods as taught in Step 6, the yield of L-theanine is about 0.8% by mass weight based on the original green tea leaf feedstock which is greater than 55% of the L-theanine present in the original feedstock. A theanine purity of 90% by mass weight of the purified theanine extract fraction may be achieved using these methods.

Food and Medicaments

As a form of foods of the present invention, there may be formulated to any optional forms, for example, a granule state, a grain state, a paste state, a gel state, a solid state, or a liquid state. In these forms, various kinds of substances conventionally known for those skilled in the art which have been allowed to add to foods, for example, a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller, etc. may be optionally contained. An amount of the elderberry extract to be added to foods is not specifically limited, and for example, it may be about 10 mg to 5 g, preferably 50 mg to 2 g per day as an amount of take-in by an adult weighing about 60 kg.

In particular, when it is utilized as foods for preservation of health, functional foods, etc., it is preferred to contain the effective ingredient of the present invention in such an amount that the predetermined effects of the present invention are shown sufficiently.

The medicaments of the present invention can be optionally prepared according to the conventionally known methods, for example, as a solid agent such as a tablet, a granule, powder, a capsule, etc., or as a liquid agent such as an injection, etc. To these medicaments, there may be formulated any materials generally used, for example, such as a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller.

An administration amount of the effective ingredient (green tea extract) in the medicaments may vary depending on a kind, an agent form, an age, a body weight or a symptom to be applied of a patient, and the like, for example, when it is administrated orally, it is administered one or several times per day for an adult weighing about 60 kg, and administered in an amount of about 10 mg to 5 g, preferably about 50 mg to 2 g per day. The effective ingredient may be one or several components of the green tea extract.

Methods also comprise administering such extracts more than one time per day, more than two times per day, more than three times per day and in a range from 1 to 15 times per day. Such administration may be continuously, as in every day for a period of days, weeks, months, or years, or may occur at specific times to treat or prevent specific conditions. For example, a person may be administered green tea species extracts at least once a day for years to enhance mental focus, cognition, and memory, or to prevent and treat type 2 diabetes mellitus, to prevent cardiovascular disease stroke, or to treat gastro-intestinal disorders, or to treat inflammatory disorders and arthritis including gout, or to treat the common cold, bacterial and fungal infections.

The foregoing description includes the best presently contemplated mode of carrying out the present invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally accepted usage by those skilled in the art. Patent and patent applications or references cited herein are all incorporated by reference in their entireties.

### EXEMPLIFICATION

### Materials

Botanicals: Four types of Chinese green tea and one type of Japanese green tea were used in this invention.

F1: Chinese green tea leaf were purchased from Nam Wan Tea Co Pte Ltd, Singapore.

F2: high grade Chinese green tea “BaifuTea” produced by Jiangsu Province, China.

F3: high grade Chinese green tea “Kai Hua Long Ding” produced by Zhejing Kai Hua Co. and collected in the spring.

F4: high grade Chinese green tea “Kai Hua Long Ding” produced by Zhejing Kai Hua Co. and collected in the autumn.

JPGT: high grade Japanese green tea.

TABLE 19
Active components of Green tea*.

| | C (wt %) | | | | |
|---|---|---|---|---|---|
| Active component | F1 | F2 | F3 | F4 | JPGT |
| volatile oil (hexane extracts) | 1.6 | 0.3 | 0.2 | 0.4 | 1.1 |
| (−)-Epigallocatechin gallate | 3.57 | 5.87 | 4.66 | 8.46 | 3.11 |
| (EGCG) | | | | | |
| (−)-Epigallocatechin (EGC) | 2.51 | 1.14 | 0.86 | 2.39 | 2.43 |
| (−)-Epicatechin gallate (ECG) | 0.68 | 1.45 | 1.36 | 1.95 | 0.55 |
| (+)-catechin | 0.15 | 0.40 | 0.31 | 0.14 | 0.24 |
| Total catechins | 6.92 | 8.85 | 7.18 | 12.94 | 6.33 |
| caffeine | 1.25 | 2.33 | 2.36 | 3.27 | 2.21 |
| Theobromine | 0.02 | 0.21 | 0.23 | 0.32 | 0.06 |
| Chlorogenic acic | 0.10 | 0.58 | 0.67 | 0.34 | N/A |
| L-theanine | 0.67 | 3.62 | 3.40 | 2.42 | 1.62 |
| Tannin acid | 0.59 | 0.60 | 0.51 | 0.61 | 0.13 |
| polysaccharide | 0.5 | | | 5.2 | 8.5 |
| Oxalic acid | 0.1 | 0.5 | 0.5 | 1.8 | 0.3 |

*Essential oil was estimated by ultrasonic extraction 1 g of feedstock in hexane for 2 hours; Catechins, caffeine, caffeine, theobromine and chlorogenic acid were extimated by ultrasonic extraction 1 g of Feedstock in methanol for 2 hours; L-theanine and Oxalic acid was estimated by ultrasonic extraction 1 g of feedstock in water for 2 hours for 2 hours.

Organic Solvents
Acetonitrile (75-05-8), for HPLC, gradient grade>99.9% (GC) (000687); Hexane (110-54-3), 95+%, spectrophotometric grade (248878), Ethanol, denatured with 4.8% isopropanol (02853); Ethanol (64-17-5), absolute, (02883); Methanol (67-56-1), 99.93%, ACS HPLC grade, (4391993); and Water (7732-18-5), HPLC grade, (95304); all were purchased from Sigma-Aldrich Co.
Acids and Bases:
Formic acid (64-18-6), 50% solution (09676); Phenol (108-95-2) (P3653); Sulfuric acid (7664-93-9), ACS reagent, 95-97% (44719); Trifluoroacetic acid (76-05-1), 99.8% spectrophotometric grade (302031) Phosphoric acid (7664-38-2), 85% solution in water (438081); all were purchased from Sigma-Aldrich Co. Potassium phoaphate, monobasic (7778-77-0), >99% purity (205925000, Lot#: A019842601) was purchased from Acros Organics Co.
Chemical Reference Standards:

(+)-catechin (154-23-4), purity 95% (03310); (−)-epicatechin (490-46-0), purity 93.6% (05125); (−)-epicatechin gallate (1257-08-5), purity 99% (05135); (−)-epigallocatechin (970-74-1), purity 98.3% (05145); (−)-epigallocatechin gallate (989-51-5), purity 94% (05151); L-theanine (3086-61-6), purity: 99.0% (20250-001); all were purchased from Chromadex (www.chromadex.com). Caffeine (58-0802), purum, anhydrous, >99% (27600); Theobromine (83-67-0), purity>99%, (T4500); and Chlorogenic acid (327-97-9), minimum 95% titration (C3878) were purchased from Sigma-Aldrich Co. Dextran standard 5000 (00269), 50,000 (00891) and 410,000 (00895) certified according to DIN were purchased from Fluka Co. Oxalic acid (144-62-7), 98% purity (194131) was purchased from Sigma-Aldrich Co. The structures of standards are shown in Table 20.

TABLE 20
Physical properties of chemical reference standards for green tea.

| | | | | | Melting |
|---|---|---|---|---|---|
| | | | Molecular | Molecular | point |
| Compound | Structure | CAS # | formular | weight | (° C.) |
| (+)- catechin | 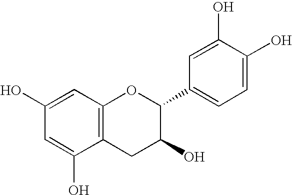 | 154-23-4 | C₁₅H₁₄O₆ | 290 | |
| (−)-EC | 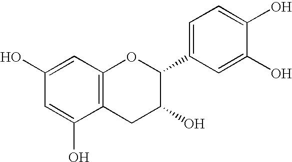 | 490-46-0 | C₁₅H₁₄O₆ | 290 | 242 |
| (−)-ECG | 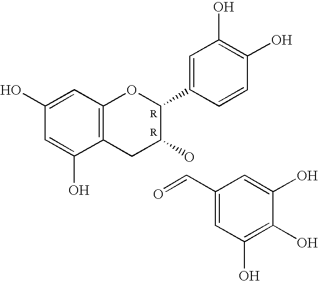 | 1257-08-5 | C₂₂H₁₈O₁₀ | 442 | |
| (−)-EGC | 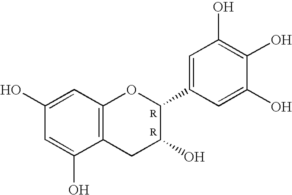 | 970-74-1 | C₁₅H₁₄O₇ | 306 | |
| (−)-EGCG | 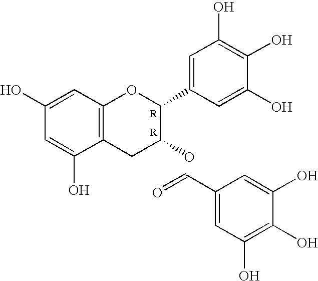 | 989-51-5 | C₂₂H₁₈O₁₁ | 458.40 | 218 |
| Caffeine | 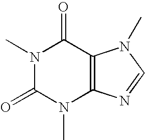 | 58-08-2 | C₈H₁₀N₄O₂ | 194 | 238 |
| Theobromine | 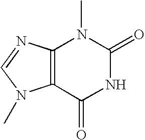 | 83-67-0 | C₇H₈H₄O₂ | 180 | 290-295 |
| Chlorogenic acid | 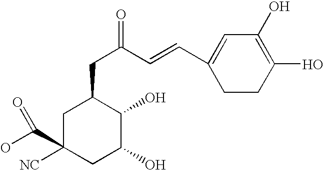 | 327-97-9 | C₁₆H₁₈O₉ | 354 | 207 |
| Theanine | 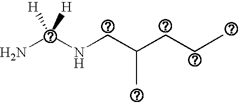 | 3081-61-6 | C₇H₁₄N₂O₃ | 174 | |
| Oxalic acid | 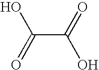 | 144-62-7 | C2H2O4 | 90 | 189 |

[symbol]

Methods
HPLC Methods
Catechin and Alkaloid Analysis.

Chromatographic system: Shimadzu high Performance Liquid Chromatographic LC-10AVP system equipped with LC10ADVP pump with SPD-M 10AVP photo diode array detector. The extraction products obtained were measured on a reversed phase Jupiter C18 column (250×4.6 mm I.D., 5μ, 300 Å) (Phenomenex, Part #: 00G-4053-E0, serial No: 2217520-3, Batch No.: 5243-17). The mobile phase consisted of A (0.5% (v/v) formic acid aqueous solution) and B (acetonitrile). The gradient was programmed as follows: within the first 6 min, A maintain at 100%, 6-10 min, solvent B increased linearly from 0% to 12%, and 10-35 min, B linear from 12% to 21%, then 35-40 min, B linear from 21% to 25%, and then 40-50 min, B linear to 100%. The injection volume was 10 μl and the flow rate of mobile phase was 1 ml/min. The column temperature was 50° C.

Methanol stock solutions of C (catechin), EGC (epigallocatechin), ECG (epicatechin gallate), EGCG (epigallocatechin gallate), caffeine, theobromine, chlorogenic acid were prepared at concentration of 1 mg/ml. One milliliter aliquots of standard solution were transferred into a 10 ml volumetric flask to yield a mixed standard solution. The mixed reference standard solution was then diluted step by step to yield a series of solutions at final concentrations of 0.5, 0.2, 0.1, 0.05, and 0.01 mg/ml, respectively. The standard curves were prepared over these five concentrations and peak area was plotted against the corresponding concentrations using linear regression to generate the standard curve. The results are summarized in Table 21.

TABLE 21
HPLC analysis results on green tea reference standards at concentration of 0.1 mg/ml
in methanol.

| | Area = m1 + m2 < weight | | | | | | | Start | Stop | |
|---|---|---|---|---|---|---|---|---|---|---|
| | (μg) | | | Retention | Area | Height | Width | time | time | Theoretical |
| ID | M1 | M2 | R² | time (min) | (mAu · min) | (mAu) | (min) | (min) | (min) | plate¹ |
| Theobromine | 22819 | 3505900 | 1.000 | 12.56_0.09 | 391030 | 47441 | 0.75 | 12.32 | 13.07 | 4468 |
| (−)-EGC | 16878 | 260560 | 0.977 | 13.59_0.05 | 45490 | 4260 | 0.38 | 13.38 | 13.76 | 20431 |
| (+)-C | 13752 | 641720 | 0.998 | 14.10 = 0.05 | 80296 | 10136 | 0.31 | 13.92 | 14.23 | 33007 |
| CA | 12396 | 1082600 | 0.999 | 14.40 = 0.04 | 124438 | 18340 | 0.38 | 14.23 | 14.61 | 22941 |
| Caffeine | 13815 | 2886100 | 0.999 | 14.79 = 0.03 | 307500 | 52704 | 0.38 | 14.61 | 15 | 24182 |
| (−)-EGCG | 22089 | 1393500 | 0.998 | 15.18 = 0.04 | 165353 | 20787 | 0.48 | 15 | 15.48 | 15977 |
| (−)-ECG | 20489 | 2184400 | 0.998 | 18.52_0.09 | 284835 | 21471 | 1.48 | 18.05 | 19.53 | 2490 |
| L-theanine | −220773 | 320873 | 0.9803 | 5.99 = 0.03 | 196406 | 15799 | 0.7 | 5.74 | 6.43 | 1177 |
| Oxalic acid | 1289 | 26066 | 0.9939 | 3.23 = 0.02 | 278335 | 30415 | 0.48 | 3.06 | 3.54 | 711 |

¹Theoretical plates was calculated by: N = 16 × (t_{R}/w)². t_{R} is retention time and w is width of the peak, https://www.mn-net.com/web%5CMN-WEB-HPLCKatalog.nsf/WebE/GRUNDLAGEN Theanine analysis.

Theanine analyses were performed on a reversed phase Jupiter C18 column (250×4.6 mm I.D. 5μ, 300 Å) (Phenomenex, Part #: 00G-4053-E0, serial No: 2217520-3, Batch No.: 5243-17). The mobile phase was water regulated with trifluoroacetic acid at concentration of 0.1%. The flow rate of the mobile phase was 1 ml/min. The detector was set at wavelength of 203 nm.

Oxalic Acid Analysis

Oxalic acid analyses were performed on a reversed phase Jupiter C18 column (250×4.6 mm I.D. 5μ, 300 Å) (Phenomenex, Part #: 00G-4053-E0, serial No: 2217520-3, Batch No.: 5243-17). The mobile phase consisted of A (0.5% KH2PO4 (w/v) aqueous solution) and B (acetonitrile). The mobile phase of 0.5% KH2PO4 (w/v) aqueous solution was prepared by dissolving solid KH2PO4 in distilled water. Then, it was adjusted to PH 2.80 with a solution of 1.0 mol/L H3PO4. The gradient was programmed as follows: solvent B increased linearly from 10% to 40% in 15 minutes and then decrease from 40% to 10% in another 5 minutes. The injection volume was 10 μl and the flow rate of mobile phase was 1 ml/min. The column temperature was 25° C. The detected wavelength was 262 nm. Different concentration of oxalic acid in water from 0.1 mg/ml to 10 mg/ml was assayed. The standard curves were prepared over these concentrations and peak area was plotted against the corresponding concentrations using linear regression to generate the standard curve. The contents of oxalic acid in the sample solution were quantified by comparing peak area in the sample solution with that of known standards.

GC-MS Methods

GC-MS analyses were performed using a Shimadzu GCMS-QP2010 system. The system includes a high-performance gas chromatograph, direct coupled GC/MS interface, electro impact (EI) ion source with independent temperature control, quaderupole mass filter et al. The system is controlled with GCMS solution Ver. 2 software for data acquisition and post run analysis. Separation was carried out on a Agilent J&W DB-5 fused silica capillary column (30 m×0.25 mm i.d., 0.25 μm film thickness) (catalog: 1225032, serial No: U.S. Pat. No. 5,285,774H) using the following temperature program. The initial temperature was 60° C., held for 1 min, then it increased to 180° C. at rate of 3° C./min, held for 35 min with total running time of 76 minutes. The sample injection temperature was 220° C. and 1 μl of sample was injected by auto injector at splitless mode in 1 minute. The carrier gas was helium and the flow rate was controlled by pressure at 40.1 KPa. Under such pressure, the flow rate was 0.79 ml/min and linear velocity was 32.5 cm/min. MS ion source temperature was 230° C., and GC/MS interface temperature was 230° C. MS detector was scanned between m/z of 50 and 500 at scan speed of 1000 AMU/second. Solvent cut off temperature was 3.5 min.

Polysaccharide Analysis

Spectrophotometer system: Shimadzu UV-1700 ultraviolet visible spectrophotometer (190-1100 nm, 1 mm resolution) has been used in this study. Colorimetric method (Dubois, M., Gilles, K. A., Hamilton, J. K., Rebers, P. A. and Smith, F., Colorimetric Method for Determination of Sugars and related substances, Analytical chemistry, 1956, 28(3), 350-356) has been used for polysaccharide analysis. Make 0.1 mg/ml stock dextran (Mw=5000, 50,000 and 410,000) solutions. Take 0.08, 0.16, 0.24, 0.32, 0.40 ml of stock solution and make up volume to 0.4 ml with distilled water. Then add in 0.2 ml 5% phenol solution and 1 ml concentrated sulfuric acid. The mixtures were allowed to stand for 10 minutes prior to performing UV scanning. The maximum absorbance was found at 488 nm. Then set the wavelength at 488 nm and measure absorbance for each sample. The results are shown in Table 22. The standard calibration curves were obtained for each of the dextran solutions as follows: Dextan 5000, Absorbance=0.01919+0.027782C (μg), R²=0.97 (N=5); Dextan 50,000, Absorbance=0.0075714+0.032196C (μg), R²=0.96 (N=5); and Dextan 410,000, Absorbance=0.03481+0.036293C (μg), R²=0.98 (N=5).

TABLE 22
Colorimetric analysis on dextran standard.

| | Dextran | Distill | 5% phenol | Sulfuric | Abs | Abs | |
|---|---|---|---|---|---|---|---|
| Tube | solution (ml) | water (ml) | (ml) | acid (ml) | (Mw = 5K) | (Mw = 50K) | Abs (Mw = 410 K) |
| Blank | 0 | 0.40 | 0.2 | 1 | 0 | 0 | 0 |
| 1 | 0.08 | 0.32 | 0.2 | 1 | 0.238 | 0.301 | 0.335 |
| 2 | 0.16 | 0.24 | 0.2 | 1 | 0.462 | 0.504 | 0.678 |
| 3 | 0.24 | 0.16 | 0.2 | 1 | 0.744 | 0.752 | 0.854 |
| 4 | 0.32 | 0.08 | 0.2 | 1 | 0.907 | 1.045 | 1.247 |
| 5 | 0.40 | 0.00 | 0.2 | 1 | 1.098 | 1.307 | 1.450 |

DART-MS Analysis of Green Tea Extracts

A JEOL AccuTOF-DART mass spectrometer (Jeol USA, Peabody, Mass.) was used in the mass spectrometric analysis of green tea extracts. This Time-of-Flight (TOF) mass spectrometer technology requires no (or minimal) sample preparation and yields masses with accuracies to 0.00001 mass units. For positive ion mode (DART+), the needle voltage was set to 3500V, heating element to 300° C., Electrode 1 to 150V, Electrode 2 to 250V, and helium gas flow to 3.69 Liters per minute (LPM). For the mass spectrometer, the following settings were loaded: Orifice 1 set to 20V, Ring Lens voltage set to 5V, and Orifice 2 set to 5V. The peaks voltage was set to 1000V in order to give resolving power starting at approximately 100 m/z. The microchannel plate detector (MCP) voltage was set at 2550V. Calibrations were performed internally with each sample using a 10% solution of PEG 600 which provided mass markers throughout the required mass range 100-1000 mass units.

The green tea samples were introduced into the DART helium plasma as powders using the closed end of a borosilicate glass melting point capillary tube. The sample collects as a thin-film on the capillary tube allowing a homogenous surface area to be exposed to the He plasma beam which maximizes delivery into the TOF. The capillary tube is held in the He plasma for approximately 3-5 seconds per analysis. No pyrolysis of the sample was seen during analysis.

For negative ion mode (DART−), the DART and AccuTOF-MS were switched to negative ion mode. The needle voltage was 3500V, heating element 300° C., Electrode 1-150V, Electrode 2-250V, and helium gas flow 3.69 LPM. For the mass spectrometer, the following settings were loaded: Orifice 1 set to −20V, ring lens voltage set to −5V, and orifice 2 set to −5V. The peaks voltage was set at 600V, to achieve appropriate resolving power at lower m/z ranges in the negative ion mode. The MCP voltage was set at 2600V. Samples were introduced into the DART in the exact same manner as in positive ion mode. Calibrations were performed internally with each sample using a solution of perfluorinated carboxylic acids.

Molecular formulas were confirmed by elemental composition and isotope matching programs provided with the JEOL AccuTOF DART-MS. A searchable database of green tea constituents was developed based upon literature. All chemical identifications (identified and unidentified) in the mass spectra are assigned with a confidence level greater than 90%.

### Example 1

### Example of Step 1

### Single Step SFE Extraction and Purification of Green Tea Essential Oil

All SFE extractions were performed on SFT 250 (Supercritical Fluid Technologies, Inc., Newark, Del., USA) designed for pressures and temperatures up to 690 bar and 200° C., respectively. This apparatus allows simple and efficient extractions at supercritical conditions with flexibility to operate in either a dynamic or static mode. This device consists of three modules: an oven, a pump and control and a collection module. The oven has one preheat column and one 100 ml extraction vessel. The pump module is equipped with a compressed air-driven pump with constant flow capacity of 300 ml/min. The collection module is a glass vial of 40 ml, sealed with caps and septa for the recovery of extracted products. It is further provided with micrometer valves and a flow meter. Extraction vessel pressure and temperature are monitored and controlled within +/−3 bar and +/−1° C.

In a typical experimental example, 25 grams of tea cut green tea leaves with size above 105 μm sieved by 140 mesh screen was loaded into a 100 ml extraction vessels for each experiment. The oven was preheated to the desired temperature before the packed vessel was loaded. After the vessel was connected into the oven, the extraction system was tested for leakage by pressurizing the system with CO₂ (850 psig), and purged. The system was closed and pressurized to desired extraction pressure using the air-driven liquid pump. The system was then left for equilibrium for ˜3 min. A sampling vial (40 ml) was weighed and connected to the sampling port. The extraction was started by flowing CO₂ at a rate of 5 SLPM (9.8 g/min), which is controlled by a meter valve. The solvent/feed ratio, defined as the weight ratio of total CO₂ used to the weight of loaded raw material, was calculated. During the extraction process, the extracted sample was weighed every 5 min. Extraction was presumed to be finished when the weight of the sample did not change more than 5% between two weighing measurements. The yield was defined to be the weight ratio of total exacts to the feed of raw feedstock material.

In this experimental example, the extraction conditions were set wherein the temperature was set at 40° C. and the pressure was set at 200 bar. The CO2 flow rate was 9.8 g/min.

### Example 2

### Example of Step 2

### Single Step SFE Decaffeination of Green Tea Plant Material

All SFE extractions were performed on SFT 250 (Supercritical Fluid Technologies, Inc., Newark, Del., USA). In a typical experimental example, the residue of the 25 grams of the essential oil extracted tea cut green tea leaves wet with 25 gm of distilled water co-solvent was loaded into a 100 ml extraction vessels for each experiment. The oven was preheated to the desired temperature before the packed vessel was loaded. After the vessel was connected into the oven, the extraction system was tested for leakage by pressurizing the system with CO₂ (850 psig), and purged. The system was closed and pressurized to desired extraction pressure using the air-driven liquid pump. The system was then left for equilibrium for ˜3 min. A sampling vial (40 ml) was weighed and connected to the sampling port. The extraction was started by flowing CO₂ at a rate of 5 SLPM (9.8 g/min), which is controlled by a meter valve. 3 ml of co-solvent was dosed into the system every time minutes. The extraction time was 4 hours. The solvent/feed ratio, defined as the weight ratio of total CO₂ used to the weight of loaded raw material, was calculated. The yield was defined to be the weight ratio of total exacts to the feed of raw feedstock material. The extraction conditions were set at 70° C. and 500 bar.

### Example 3

### Example of Step 3

### 95% Ethanol Leaching Extraction

Typical examples of 2 stage solvent extractions of the catechin chemical constituents of green tea leaf material is as follows: The feedstock was 25 gm of tea cut green tea leaf SFE residue from Step 2 SCCO2 decaffeination or raw green tea leaf feedstock. The solvent was 250 ml of 95% ethanol. In this method, the feedstock material and 250 ml 95% ethanol were separately loaded into 500 ml extraction vessel and mixed in a heated water bath at 70° C. for 2 hours. The extraction solution was filtered using Fisherbrand P4 filter paper having a particle retention size of 4-8 μm, centrifuged at 3000 rpm for 20 minutes, and the particulate residue used for further extraction. The filtrate (supernatant) was collected for yield calculation and HPLC analysis. The residue of Stage 1 was extracted for 2 hours (Stage 2) using the aforementioned methods. The supernatant extracts were combined and the ethanol removed using a rotary evaporator. If further purification of the catechin fraction is desired, then the alcohol free crude catechin extraction product is dissolved the 250 ml of distilled water for Step 4 processing. The residue of Stage 2 extraction was same for further processing for theanine and polysaccharide fractions (see Step 5).

### Example 4

### Examples of Step 4 Affinity Adsorbent Extraction of Catechin Fractions

In typical experiments, the working solution was the transparent aqueous solution of the green tea two-stage 95% ethanol leaching extract in Step 3. For these examples, 25 gm green SFE decaffeinated residue was two-stage leaching extracted using 250 ml of 95% ethanol at 70° C. (solvent feed ratio 20/1) as described in Step 3. The two-stage extracts were combined and ethanol was removed using rotary evaporation. Distilled water was then added to reconstitute the original concentration of the solution (500 ml volume).

The affinity adsorbent polymer resin was XAD7HP (see Appendix 1). 30 gm of affinity adsorbent was pre-washed with 95% ethanol (4-5 BV) and distilled water (4-5 BV) before and after packing into a glass column with an ID of 10 mm and length of 350 mm. The BV (bed volume) of adsorbent was 35 ml. 100 ml aqueous solution (de-ethanolized step 3 leaching solution) having a concentration in solution=4.8-9.6 mg/ml was loaded into the packed column at a flow rate of 1.2 ml/min (2 BV/hr). The loading time was 85 minutes. The loaded column was washed with 150 ml of distilled water at a flow rate of 10 BV/hr with a washing time of 25 minutes. To decaffeinate the loaded column, 100 ml of 5% H2SO4 in 10% ethanol was used to elute the caffeine compounds at a flow rate of 2.2 ml/min (2 BV/hr). The eluate was discarded. Then 250 ml of distilled water was used to wash the column at a flow rate of 6 ml/min (12 BV/hr) or until the washings solutions became neutral pH. 100 ml of 80% ethanol was used to elute the catechins from the loaded column at a flow rate of 1.2 ml/min (2 BV/hr) with an elution time of 85 minutes. During the elution, 6 fractions (F1-F6) were collected at 0-0.7 (F1), 0.8-1.0 (F2), 1.0-1.1 (F3), 1.1-1.3 (F4), 1.3-1.6 (F5), and 1.6-3 (F6) BV, respectively. Then 3 BV of absolute ethanol was used to clean out the remaining chemicals on the column at a flow rate of 3.6 BV/hr followed by washing with 3 BV distilled water at 3.8 BV/hr. The loading, effluent, washings, and caffeine eluate were all collected, measured for mass content and analyzed using HPLC to measure the catechins (EGCG, EGC, ECG, C), caffeine, theobromine, and chlorogenic acid. Each elution fraction was collected and analyzed by HPLC.

### Example 5

### Example of Step Water Leaching Process for Extraction of Polysaccharides and Theanine

In a typical experiment example of the water leaching process, 20 gm of residue from the 95% ethanol leaching process of Step 3 and 400 ml of distilled water were separately loaded into a 500 ml extraction vessel and mixed in a water bath at 70° C. for 2 hours. The top clear layer was decanted and a second stage extraction of the solid residue was extracted with 400 ml of distilled water using the same methods. The two-stage extraction solutions were centrifuged at 2000 rpm for 10 minutes and filtered with Fisherbrand P4 paper (particle retention size of 4-8 μm). The two-stage decanted supernatant solutions were collected and combined for yield calculation and HPLC analysis for theanine content.

### Example 6

### Example of Step 6 Polysaccharide Fraction and Theanine Extraction and Purification

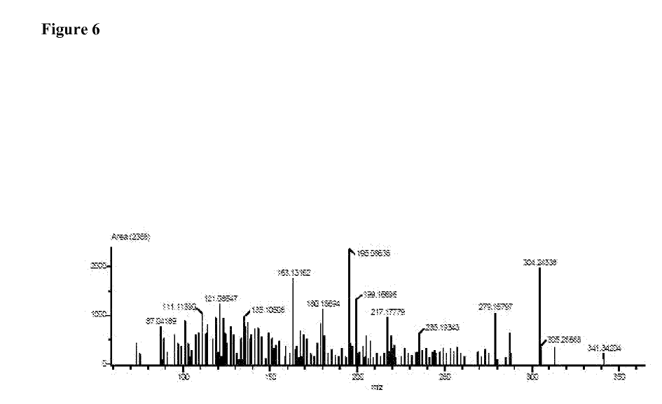

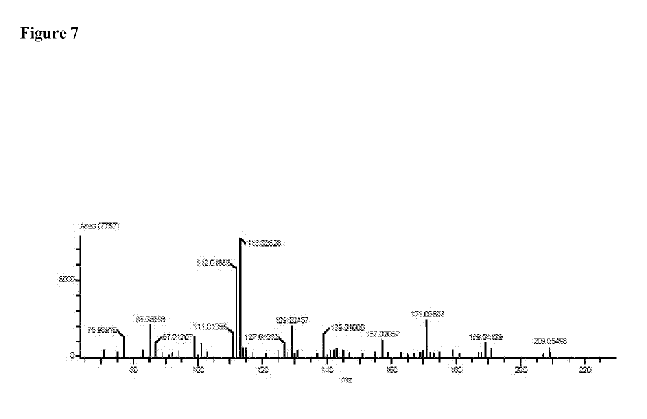

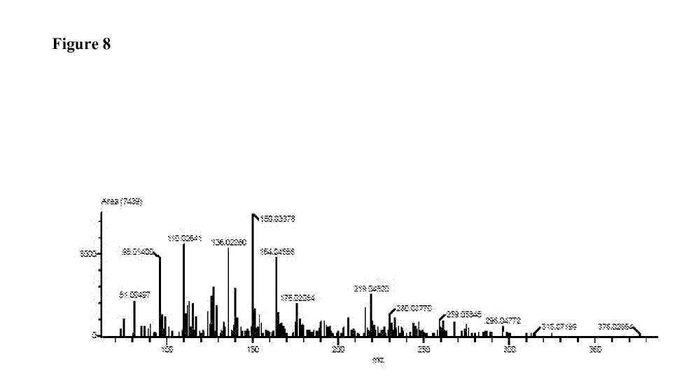

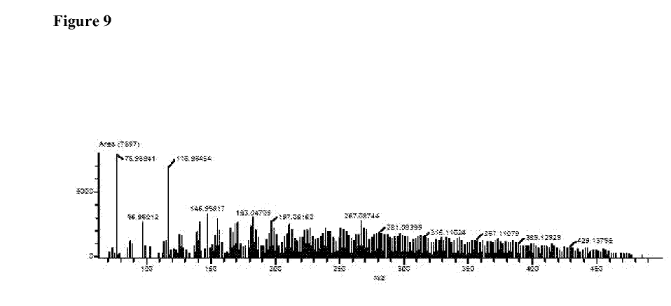

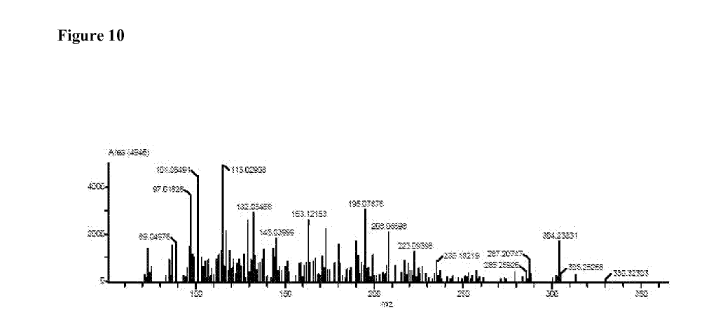

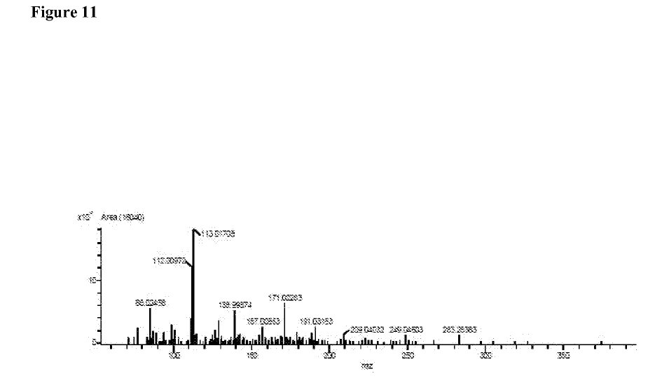

A typical experimental example of solvent extraction and precipitation of the water soluble, ethanol insoluble purified polysaccharide fraction chemical constituents and the theanine chemical constituents of green tea is as follows: 20 gm of the solid residue from the 2 stage 95% ethanol leaching extraction of Step 3 was extracted using 2 stage distilled water leaching as described above in Step 5. The Step 5 two stage extract solutions were combined. Vacuum rotary evaporation was used to concentrate the clear supernatant extract solution removing 60% of the water solvent. Then, anhydrous ethanol was added to make up a final ethanol concentration of 75%. This solution was allowed to sit for 1 hour and a precipitate was observed. The extraction solution was centrifuged at 2,000 rpm for 10 minutes and the supernatant decanted, freeze dried, and saved for further processing. The polysaccharide precipitate was collected and freeze dried. The dried polysaccharide fraction was weighed and dissolved in water for analysis of polysaccharide purity with the colormetric method using dextran as reference standards. Moreover, AccuTOF-DART mass spectrometry was used to further profile the molecular weights of the compounds comprising the polysaccharide fractions. The results are shown in FIGS. 6-11.

The dried supernatant product containing L-theanine was dissolved in distilled water to make a 10% solution. To this solution, 4 volume of absolute ethanol was added, mixed, and allowed to stand for 1 hour. The solution was then centrifuged at 6,000 rpm for 10 minutes and decanted. The precipitates were discarded. The supernatant solution collected was concentrated using vacuum rotary evaporation at 60° C. to an 80% ethanol solution. This 80% ethanol solution was allowed to cool to room temperature. Then, 4 volume of ethanol was added to the solution. This solution was placed in a refrigerator at 0° C. for 24 hours for crystallization of the theanine compound. The solution was centrifuged at 2000 rpm for 10 min and the crystals were then collected and dried at 60° C. under vacuum.

### Example 7

### The Following Ingredients are Mixed for the Formulation

| | |
|---|---|
| Extract of Green Tea | 150.0 mg |
| Essential Oil Fraction (10 mg, 7% dry weight) | |
| Catechin Fraction (90 mg, 60% dry weight) | |
| Theanine Fraction (20, 13% dry weight) | |
| Polysaccharides (30 mg, 20% dry weight) | |
| Stevioside (Extract of Stevia) | 12.5 mg |
| Carboxymethylcellulose | 35.5 mg |
| Lactose | 77.0 mg |
| Total | 275.0 mg |

The novel extract of green tea comprises purified essential oil fraction, catechin fraction, theanine fraction, and polysaccharide fraction by % mass weight greater than that found in the natural rhizome material or convention extraction products. The formulations can be made into any oral dosage form and administered daily or to 15 times per day as needed for the physiological, psychological, medical effects desired (anti-oxidant, oxygen free radical scavenging, and nitrosation inhibition activities, immunological enhancement, anti-osteoporosis, cardiovascular disease prevention and therapy, cerebrovascular disease prevention and therapy, cholesterol lowering activity, prevention and treatment of cancer, treatment of HIV and viral diseases, weight loss and thermogenesis, prevention of aging, management of diabetes mellitus, enhancement of memory and cognition, anxiety reduction, and mood enhancement).

### Example 8

### The Following Ingredients were Mixed for the Following Formulation

| | |
|---|---|
| Extract of Green Tea | 150.0 mg |
| Essential Oil Fraction (5 mg, 3% dry weight) | |
| Phenolic Acid Fraction (90.0 mg, 60% dry weight) | |
| Theanine (10.0 mg, 7% dry weight) | |
| Polysaccharides (45.0 mg, 30% dry weight) | |
| Vitamin C | 15.0 mg |
| Sucralose | 35.0 mg |
| Mung Bean Powder 10:1 | 50.0 mg |
| Mocha Flavor | 40.0 mg |
| Chocolate Flavor | 10.0 mg |
| Total | 300.0 mg |

The novel extract composition of Green tea comprises purified essential oil, catechin, theanine, and polysaccharide chemical constituent fractions by % mass weight greater than that found in the natural plant material or conventional extraction products. The formulation can be made into any oral dosage form and administered safely up to 15 times per day as needed for the physiological, psychological and medical effects desired.

### REFERENCES CITED

1. Alschuler L. Am J Nat Med 5:28-31, 1998.
2. Balentine D A et al. Crit Rev Food Sci Nutr 37:693-704, 1997.
3. Graham H N. Prev Med 21:a334-350, 1992.
4. Lin Y-L et al. J Agric Food Chem 44:1387-1394, 1996.
5. Lu H et al. Drug Metab Dispos 31:452-461, 2003.
6. Chen H et al. J Agric Food Chem 52(11): 3333-3336, 2004.
7. Chen C-W & Ho C T. J Food Lipids 2:35-46, 1995.
8. Katiyar S K et al. Carcinogenesis 14:849-855, 1993.
9. Yen G-C et al. J Agric Food Chem 43:27-32, 1995.
10. Mukhtar H M et al. Prev Med 21:351-360, 1992.
11. Wang Z Y et al. Cancer Res 52:1162-1170, 1992.
12. Brown M D. Alt Med Rev 4:360-370, 1999.
13. Katiyar S K et al. Arch Dermatol 136:989-994, 2000.
14. Sugiyama T & Sadzuka Y. Biochim Biophys Acta 1653(2):47-59, 2003.
15. Elmets C A et al. J Am Acad Dermatol 44:425-432, 2001.
16. Tsubono Y & Tsugane S. Ann Epidemiol 7:280-284, 1997.
17. Geleijnse J M et al. Arch Intern Med 159:2170-2174, 1999.
18. Egashira N et al. Neurosci Lett 363(1):58-61, 2004.
19. Kakuda T. Biol Pharm Bull 25(12): 1531-1538, 2002.
20. Krahwinkel T & Willershausen B. Brit Med J 310:693-696, 1995.
21. Hegarty V M et al. Am J Clin Nutr 71:1003-1007, 2000.
22. Kamath A B et al. Proc Natl Acad Sci USA 100:6009-6014, 2003.
23. Weisburger J H. Tea and Health. In: Herbal and Tradition Medicine (Packer, L, Ong C N, & Halliwell B, eds), Marcel Dekker, New York, 2004, pp. 117-144.
24. Zheng G et al. In Vivo 18(1):55-62, 2004.
25. Juneja L R et al. Trends Food Sci Tech 10:199-204, 1999.
26. Lu K et al. Hum Psychopharmacol 19(7):457-465, 2004.
27. Wang D W et al. J Agric Food Chem 49(1):507-510, 2001.
28. Williamson E M. Phytomedicine 8(5):401-409, 2001.
29. Xu X et al. Zhong Yao Cai 28(4):327-329, 2005.
30. Kopcak UaM & Rahoma S. J Supercritical Fluids 34:209-214, 2005.

We claim:

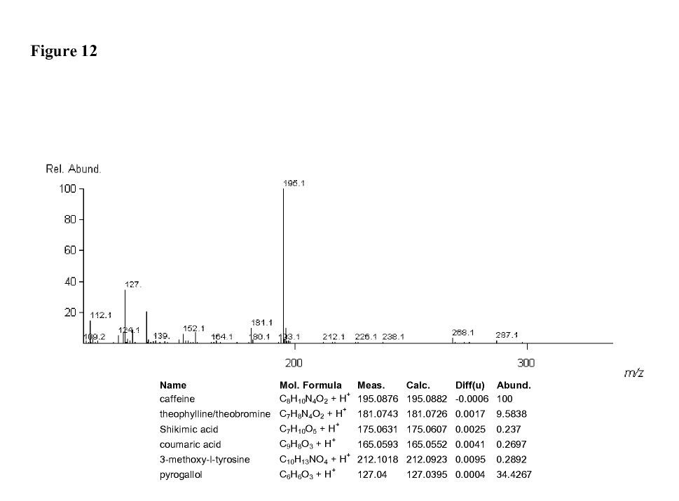

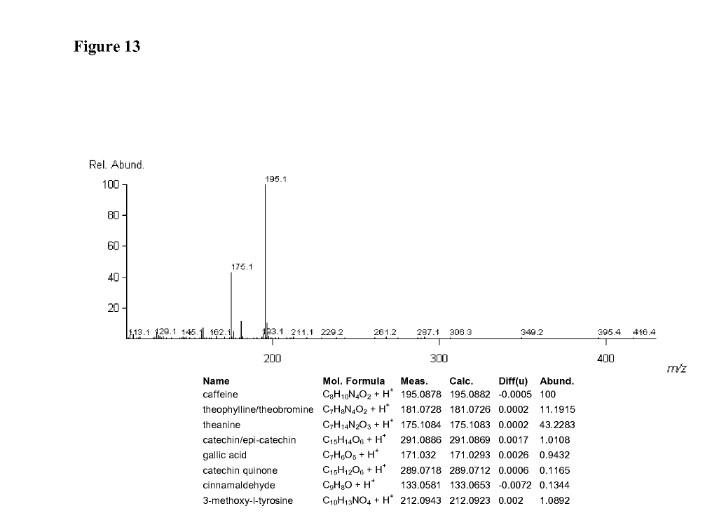

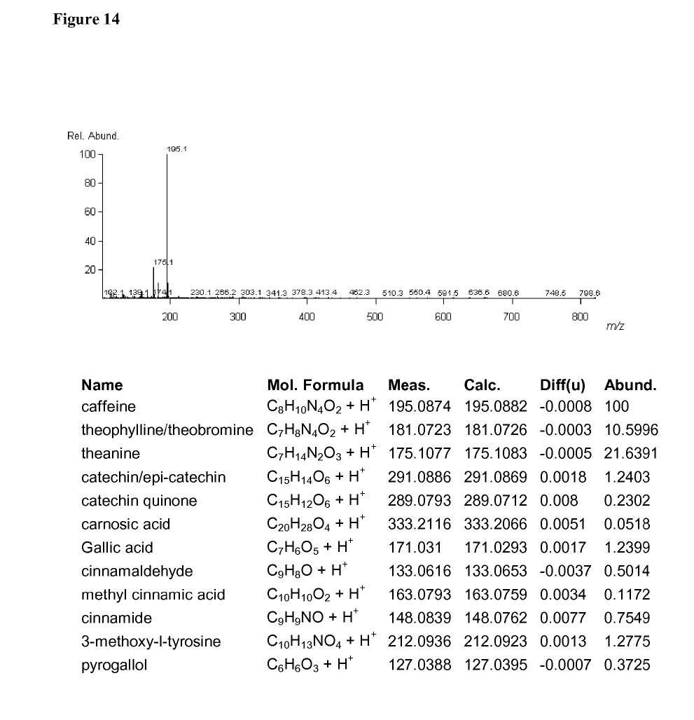

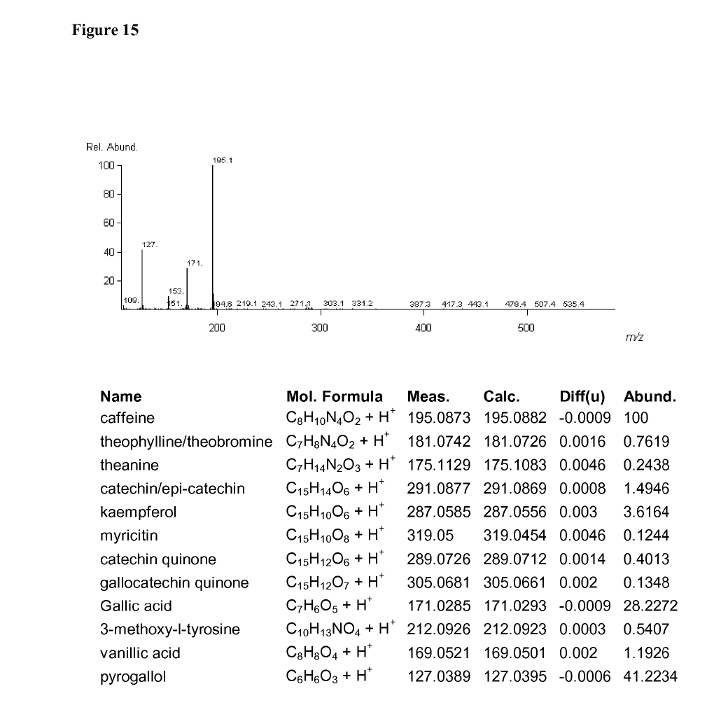

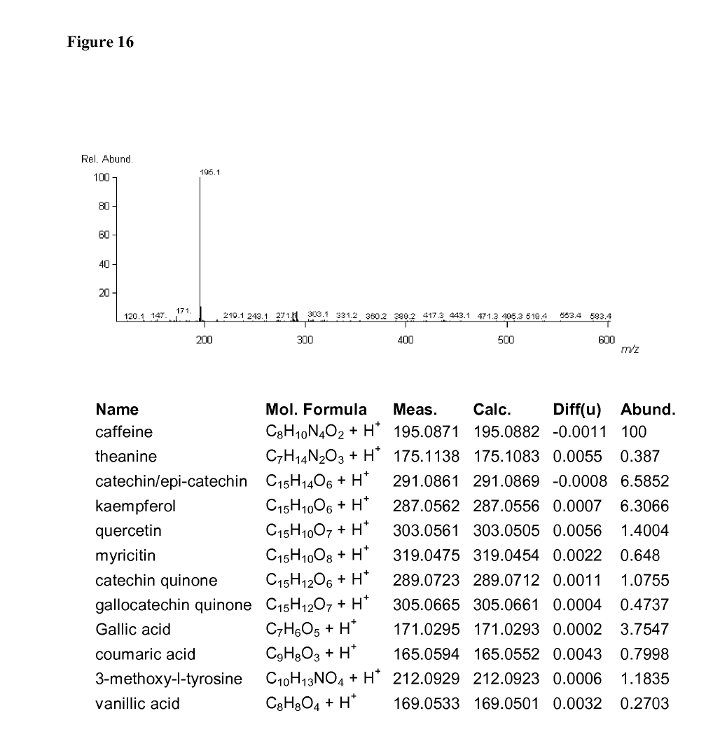

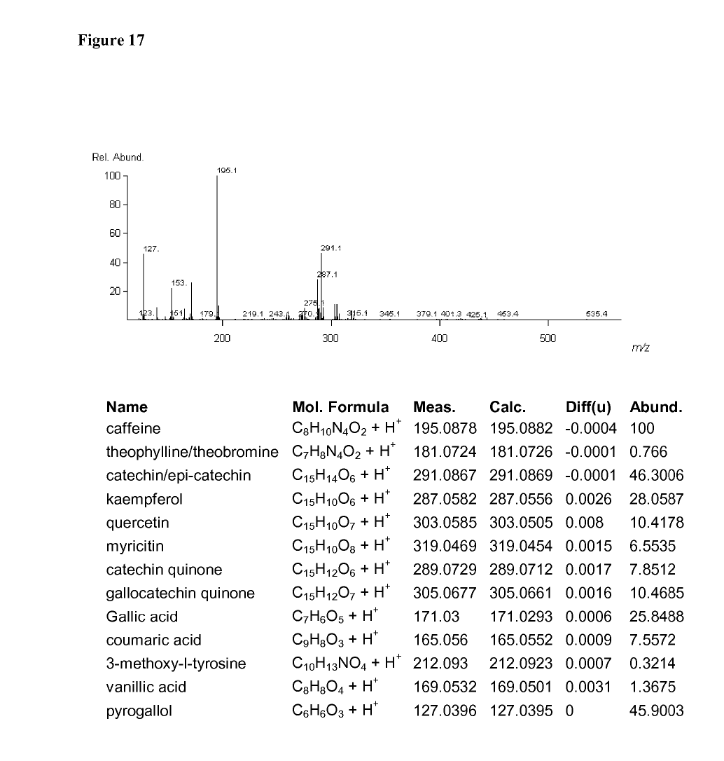

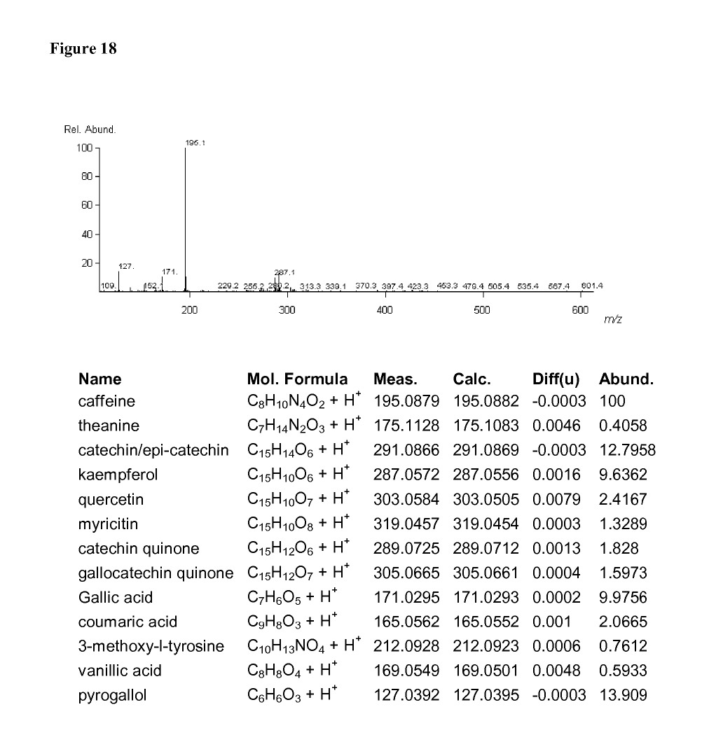

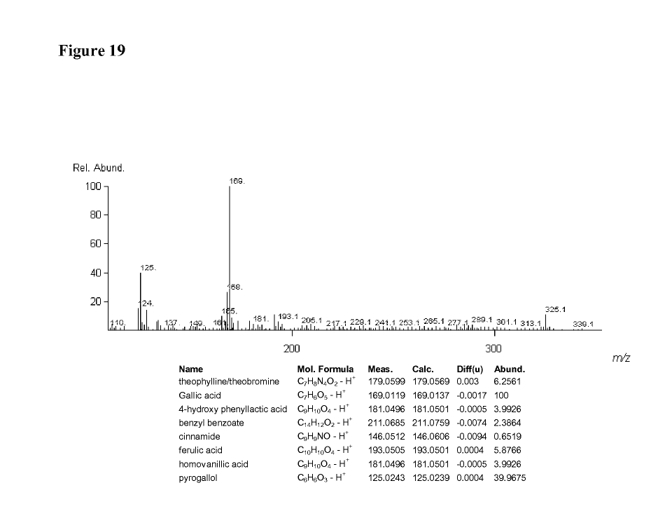

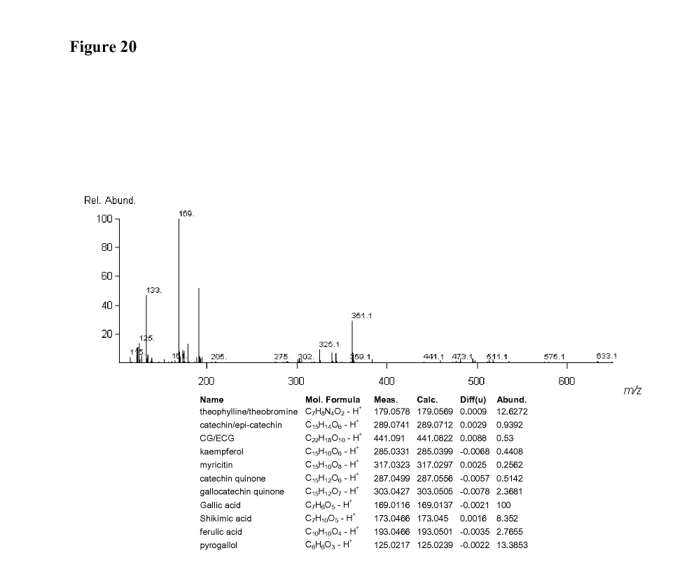

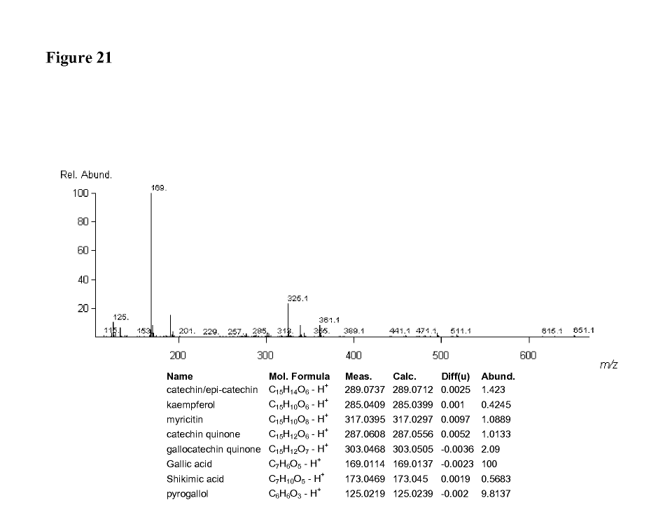

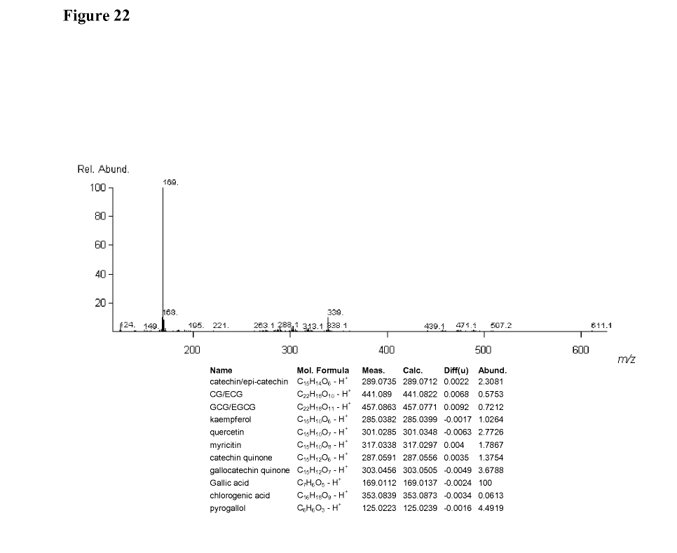

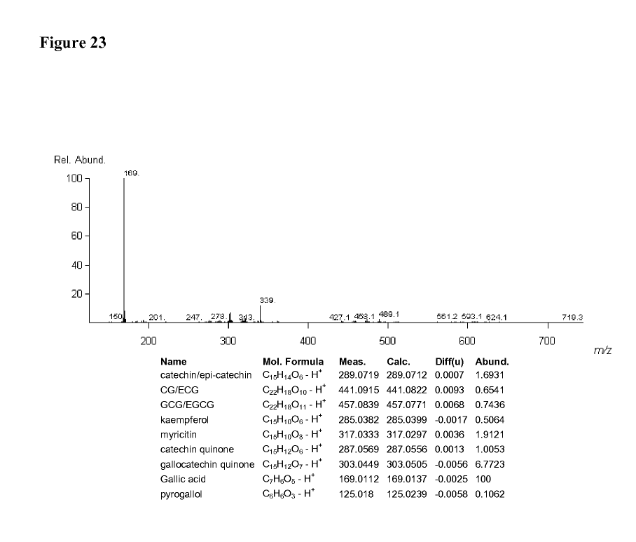

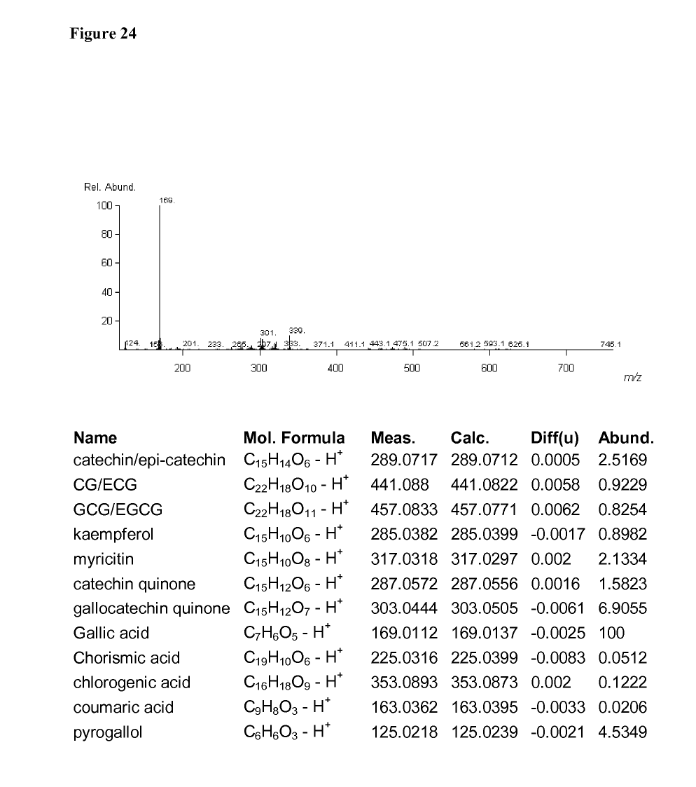

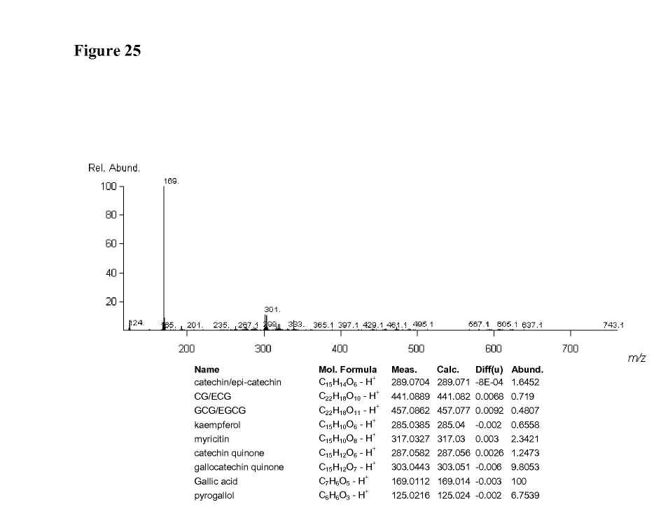

1. A green tea species extract comprising a fraction having a Direct Analysis in Real Time (DART) mass spectrometry chromatogram of any of FIGS. 6 to 25.

2. The green tea species extract of claim 1, wherein the extract comprises a compound selected from the group consisting of an essential oil, a polyphenol, a polysaccharide, and combinations thereof.

3. The green tea species extract of claim 2, wherein the essential oil is selected from the group consisting of n-hexadecanoic acid, tetradecanoic acid, 9-hexadecanol, 1-undecanol, 1-hexadecanol, oleyl alcohol, 9-octadecen-1-ol, nonadecanol, and combinations thereof.

4. The green tea species extract of claim 2, wherein the polyphenol is selected from the group consisting of catechins, flavanols, flavonol glycosides, and combinations thereof.

5. The green tea species extract of claim 4, wherein the catechin is selected from the group consisting of catechin (C), epicatechin (EC), epicatechin gallate (ECG), gallocatechin (GC), epigallocatechin gallate (EGCG), epigallocatechin (EGC), and combinations thereof.

6. The green tea species of claim 4, wherein the flavanol is selected from the group consisting of quercetin and rutin.

7. The green tea species of claim 4, wherein the flavonol glycoside is kaempferol.

8. The green tea species of claim 2, wherein the polysaccharide is selected from the group consisting of glucose, arabinose, galactose, rhamnose, xylose uronic acid and combinations thereof.

9. The green tea species of claim 1 substantially free of caffeine, oxalic acid, or tannins.

10. The green tea species of claim 2, wherein the amount of essential oil is greater than 2% by weight.

11. The green tea species extract of claim 2, wherein the amount of essential oil is from 25% to 90% by weight.

12. The green tea species extract of claim 2, wherein the amount of essential oil is from 50% to 90% by weight.

13. The green tea species extract of claim 2, wherein the amount of essential oil is from 75% to 90% by weight.

14. The green tea species extract of claim 2, wherein the amount of polyphenol is greater than 40% by weight.

15. The green tea species extract of claim 2, wherein the amount of polyphenol is from 50% to 90% by weight.

16. The green tea species extract of claim 2, wherein the amount of polyphenol is from 75% to 90% by weight.

17. The green tea species extract of claim 2, wherein the amount of polysaccharide is greater than 15% by weight.

18. The green tea species extract of claim 2, wherein the amount of polysaccharide is from 25% to 90% by weight.

19. The green tea species extract of claim 2, wherein the amount of polysaccharide is from 50% to 90% by weight.

20. The green tea species extract of claim 2, wherein the amount of polysaccharide is from 75% to 90% by weight.

21. The green tea species extract of claim 1, wherein the extract comprises an essential oil from 2% to 97% by weight, a catechin from 15% to 98% by weight, a theanine from 4% to 90% by weight, and a polysaccharide from 9% to 98% by weight.

22. Food or medicament comprising the green tea species extract of claim 1.

23. A method of preparing a green tea species extract having at least one predetermined characteristic comprising sequentially extracting a green tea species plant material to yield an essential oil fraction, a polyphenol fraction, and a polysaccharide fraction by
a) extracting a green tea species plant material by super critical carbon dioxide extraction to yield an essential oil fraction and a first residue;
b) extracting either a green tea species plant material or the first residue from step a) by alcoholic extraction to yield the polyphenolic fraction and a second residue; and
c) extracting the second residue from step b) by water extraction and precipitating the polysaccharide with alcohol to yield the polysaccharide fraction.

24. The method of claim 23, wherein the first residue from step a) is further decaffeinated by supercritical carbon dioxide extraction.

25. The method of claim 23, wherein the polyphenolic fraction is further purified by affinity adsorbent chromatography.

26. The method of claim 23, wherein step a) comprises:
1) loading in an extraction vessel ground green tea species plant material;
2) adding carbon dioxide under supercritical conditions;
3) contacting the green tea species plant material and the carbon dioxide for a time; and
4) collecting an essential oil fraction in a collection vessel.

27. The method of claim 23, further comprising the step of altering the essential oil chemical compound ratios by fractionating the essential oil fraction with a supercritical carbon dioxide fractional separation system.

28. The method of claim 26, wherein supercritical conditions comprise 60 bars to 800 bars of pressure at 35° C. to 90° C.

29. The method of claim 26, wherein supercritical conditions comprise 60 bars to 500 bars of pressure at 40° C. to 80° C.

30. The method of claim 26, wherein the time is 30 minutes to 2.5 hours.

31. The method of claim 26, wherein the time is 1 hour.

32. The method of claim 23, wherein step b) comprises:
1) contacting ground green tea species plant material or the first residue from step a) with an alcoholic solvent for a time sufficient to extract polyphenol chemical constituents;
2) passing an aqueous solution of extracted polyphenolic chemical constituents from step 1) through an affinity adsorbent resin column wherein the polyphenolic constituents are adsorbed;
3) eluting the caffeine compounds from the affinity adsorbent using an acidic elution solvent; and
4) eluting the polyphenolic chemical constituents from the affinity adsorbent resin using a hydro-alcoholic eluting solvent.

33. The method of claim 32, wherein the hydro-alcoholic solution comprises ethanol and water wherein the ethanol concentration is 10-95% by weight.

34. The method of claim 32, wherein the hydro-alcoholic solution comprises ethanol and water wherein the ethanol concentration is 25% by weight.

35. The method of claim 32, wherein step 1) is carried out at 30° C. to 100° C.

36. The method of claim 32, wherein step 1) is carried out at 60° C. to 100° C.

37. The method of claim 32, wherein the time is 1-10 hours.

38. The method of claim 32, wherein the time is 1-5 hours.

39. The method of claim 32, wherein the time is 2 hours.

40. The method of claim 23, wherein step c) comprises:
1) contacting the second residue from step b) with water for a time sufficient to extract polysaccharides; and
2) precipitating the polysaccharides from the water solution by alcohol precipitation.

41. The method of claim 40, wherein the water is at 70° C. to 90° C.

42. The method of claim 40, wherein the water is at 80° C. to 90° C.

43. The method of claim 40, wherein the time is 1-5 hours.

44. The method of claim 40, wherein the time is 2-4 hours.

45. The method of claim 40, wherein the time is 2 hours.

46. The method of claim 40, wherein the alcohol is ethanol.

47. A green tea species extract prepared by the method of claims 23.

48. A green tea species extract comprising pyrogallol, theophylline/theobromine at 25 to 35% by weight of the pyrogallol, shikimic acid at 0.1 to 5% by weight of the pyrogallol, coumaric acid at 0.1 to 5% by weight of the pyrogallol, and 3-methoxy-1-tyrosine at 0.1 to 5% by weight of the pyrogallol.

49. A green tea species extract comprising theanine, theophylline/theobromine at 20 to 30% by weight of the theanine, catechin/epicatechin at 1 to 10% by weight of the theanine, gallic acid at 1 to 10% by weight of the theanine, catechin quinone at 0.1 to 5% by weight of the theanine, cinnamaldehyde at 0.1 to 5% by weight of the theanine, and 3-methoxy-1-tyrosine at 1 to 10% by weight of the theanine.

50. A green tea species extract comprising theanine, theophylline/theobromine at 45 to 55% by weight of the theanine, catechin/epicatechin at 1 to 10% by weight of the theanine, carnosic acid at 0.1 to 5% by weight of the theanine, gallic acid at 1 to 10% by weight of the theanine, catechin quinone at 0.5 to 5% by weight of the theanine, cinnamaldehyde at 1 to 10% by weight of the theanine, methyl cinnamic acid at 0.1 to 5% by weight of the theanine, cinnamide at 1 to 10% by weight of the theanine, and 3-methoxy-1-tyrosine at 1 to 10% by weight of the theanine.

51. A green tea species extract comprising pyrogallol, theophylline/theobromine at 1 to 10% by weight of the pyrogallol, theanine at 0.1 to 5% by weight of the pyrogallol, catechin/epicatechin at 1 to 10% by weight of the pyrogallol, kaempferol at 5 to 15% by weight of the pyrogallol, myricitin at 0.1 to 5% by weight of the pyrogallol, gallocatechin quinone at 0.1 to 5% by weight of the pyrogallol, gallic acid at 65 to 75% by weight of the pyrogallol, catechin quinone at 0.5 to 5% by weight of the pyrogallol, vanillic acid at 1 to 10% by weight of the pyrogallol, and 3-methoxy-1-tyrosine at 1 to 5% by weight of the pyrogallol.

52. A green tea species extract comprising kaempferol, theanine at 1 to 10% by weight of the kaempferol, catechin/epicatechin at 95 to 105% by weight of the kaempferol, quercetin at 20 to 30% by weight of the kaempferol, myricitin at 5 to 15% by weight of the kaempferol, gallocatechin quinone at 5 to 10% by weight of the kaempferol, gallic acid at 55 to 65% by weight of the kaempferol, catechin quinone at 1 to 10% by weight of the kaempferol, coumaric acid at 10 to 20% by weight of the kaempferol, vanillic acid at 1 to 10% by weight of the kaempferol, and 3-methoxy-1-tyrosine at 15 to 25% by weight of the kaempferol.

53. A green tea species extract comprising pyrogallol, theophylline/theobromine at 0.5 to 5% by weight of the pyrogallol, catechin/epicatechin at 95 to 105% by weight of the pyrogallol, kaempferol at 55 to 65% by weight of the pyrogallol, quercetin at 20 to 30% by weight of the pyrogallol, myricitin at 10 to 20% by weight of the pyrogallol, gallocatechin quinone at 20 to 30% by weight of the pyrogallol, gallic acid at 50 to 60% by weight of the pyrogallol, catechin quinone at 15 to 25% by weight of the pyrogallol, coumaric acid at 15 to 25% by weight of the pyrogallol, vanillic acid at 1 to 10% by weight of the pyrogallol, and 3-methoxy-1-tyrosine at 0.5 to 5% by weight of the pyrogallol.

54. A green tea species extract comprising pyrogallol, theophylline/theobromine at 0.5 to 5% by weight of the pyrogallol, catechin/epicatechin at 95 to 105% by weight of the pyrogallol, kaempferol at 55 to 65% by weight of the pyrogallol, quercetin at 20 to 30% by weight of the pyrogallol, myricitin at 10 to 20% by weight of the pyrogallol, gallocatechin quinone at 20 to 30% by weight of the pyrogallol, gallic acid at 50 to 60% by weight of the pyrogallol, catechin quinone at 15 to 25% by weight of the pyrogallol, coumaric acid at 15 to 25% by weight of the pyrogallol, vanillic acid at 1 to 10% by weight of the pyrogallol, and 3-methoxy-1-tyrosine at 0.5 to 5% by weight of the pyrogallol.

55. A green tea species extract comprising pyrogallol, theanine by weight of the pyrogallol, catechin/epicatechin at 90 to 100% by weight of the pyrogallol, kaempferol at 65 to 75% by weight of the pyrogallol, quercetin at 15 to 25% by weight of the pyrogallol, myricitin at 5 to 15% by weight of the pyrogallol, gallocatechin quinone at 5 to 15% by weight of the pyrogallol, gallic acid at 65 to 75% by weight of the pyrogallol, catechin quinone at 5 to 15% by weight of the pyrogallol, coumaric acid at 10 to 20% by weight of the pyrogallol, vanillic acid at 1 to 10% by weight of the pyrogallol, and 3-methoxy-1-tyrosine at 1 to 10% by weight of the pyrogallol.
